(19) United States
(12) Patent Application Publication
(10) Pub. No.: US 2024/0156526 A1
(43) Pub. Date: May 16, 2024

(54) NEGATIVE PRESSURE BASED IMAGING AND THERAPEUTIC APPARATUS AND SYSTEM FOR WELL-CONFINED ABNORMAL MUCOSAL TISSUE ABLATION AND WORKING METHOD OF THE SYSTEM

(71) Applicants: IZMIR BIYOTIP VE GENOM MERKEZI, lzmir (TR); DOKUZ EYLUL UNIVERSITESI, lzmir (TR)

(72) Inventors: Serhat TOZBURUN, Izmir (TR); Merve TURKER BURHAN, Izmir (TR); Ender Berat ELLIDOKUZ, Izmir (TR)

(73) Assignees: IZMIR BIYOTIP VE GENOM MERKEZI, Izmir (TR); DOKUZ EYLUL UNIVERSITESI, Izmir (TR)

(21) Appl. No.: 18/276,874

(22) PCT Filed: Feb. 11, 2022
(86) PCT No.: PCT/TR2022/050113

§ 371 Date: Aug. 10, 2023

(30) Foreign Application Priority Data
Feb. 11, 2021 (TR) 2021/001949

(51) Int. Cl. A61B 18/22

(52) U.S. Cl. CPC A61B 18/22; A61B 2018/00577

(57) ABSTRACT

The invention relates to a method, system, and apparatus used in endoscopic medical diagnostic applications and endoscopic medical treatment applications, particularly in applications for imaging and treating lesions occurring in the biological mucosal layer with the thermal ablation (coagulation) method.

### TECHNICAL FIELD

The present invention relates to a method, system, and probe (i.e., endoscopic apparatus) used in endoscopic medical diagnostic applications and endoscopic medical therapy applications, particularly in imaging and treatment of lesions occurring in the mucosal layer by thermal ablation methods.

### BACKGROUND ART

The innermost layer, the mucosa layer, is critical for organs in the gastrointestinal tract. Many diseases usually begin in superficial tissues such as the mucosa layer. These diseases include esophagus cancer (e.g., esophageal adenocarcinoma), stomach cancer, colon cancer, and rectal cancer, which presents as a precancerous mucosal lesion in the gastrointestinal tract and spreads to deeper tissues.

Barrett's esophagus describes abnormal mucosal formation resulting from metaplasia of esophageal epithelial tissue, for example, due to inflammation caused by chronic acid reflux disease. This precancerous condition of the esophagus is associated with esophageal adenocarcinoma, the seventh most common type of cancer worldwide. Depending on the stages, four main methods are generally used in the treatment of Barrett's esophagus. These methods are respectively; (I) drug therapy method for mostly acid reflux treatment, (II) endoscopic mucosal resection method, (III) endoscopic submucosal dissection method, and (IV) endoscopic mucosal ablation method.

Treatment, which includes proton pump inhibitor drugs and acid regulators (e.g., antacids, H2 receptor blockers), aggressively reduces gastric acid production. However, drug therapy aims to prevent the further damage caused by gastric acid in the mucosa layer due to reflux rather than the treatment or destruction of damaged tissues.

Endoscopic mucosal resection and endoscopic submucosal dissection treatment methods show technical similarities. Both methods are based on separating the mucosal lesion from the deep tissues by fluid injection and then mechanical excision. Generally, the endoscopic mucosal resection method is used for superficial lesions smaller than 1.5 cm in diameter. In contrast, the endoscopic submucosal dissection method, which requires more extensive instruments such as an electrosurgical knife, is used for large (>1.5 cm) lesions that have spread to deep tissues.

Another traditional endoscopic intervention is endoscopic mucosal ablation. Depending on the thermal damage of the target lesion, this method can be divided into several sub-techniques; for example, multipolar electrocoagulation, argon plasma coagulation, cold therapy (cryotherapy), photodynamic therapy, radiofrequency ablation, and photothermal (laser) ablation. Multipolar electrocoagulation is a technique that provides heat-induced ablation, for example, by direct contact of tissue and electrodes and the passage of electric current.

Argon plasma coagulation is a technique for increasing target tissue temperature by non-invasively transmitting electrical energy by a flow of ionized current. Radiofrequency ablation is an ablation technique based on necrosis of the target lesion where the electrodes are in direct contact using heat generated from alternating current (˜500 kHz). Photothermal ablation is a technique based on the absorption of laser energy by the target mucosal layer and converting it into heat energy. The primary approach approximates the targeted treatment depth using laser wavelength-dependent optical penetration depth and laser fluence.

A combination of different endoscopic intervention methods may also be effective in the treatment of mucosal lesions. However, even though successful results are obtained, the fixed treatment depth produced by these endoscopic interventions, that is, insufficient or uncontrollable treatment depth, appears as a significant obstacle to the complete cleaning of mucosal lesions, the thickness of which can vary from patient to patient. Due to the uncontrolled depth of treatment, in some cases, healthy esophageal tissues under the target surface may be unintentionally thermally/mechanically damaged. Besides, side effects such as bleeding and perforation that limit endoscopic imaging may occur and may cause undesirable results such as organ narrowing and chest pain. Therefore, thermal mucosal therapy is usually scheduled for multiple sessions rather than a single session to minimize the likelihood of these side effects.

On the other hand, there are also endoscopy treatment methods with relatively more controllable side effects, such as cryotherapy and photodynamic therapy. Cryotherapy is based on rapid freezing and slow tissue thawing by low-pressure spraying liquid nitrogen to the target lesion. However, the process is repeated until sufficient thermal damage is achieved. Also, more than one treatment session may be needed over several months. Photodynamic therapy is based on labeling the target lesion with a photosensitive drug. The target lesion is destroyed without damaging the healthy tissue with the free oxygen radicals produced by the irradiated drug. However, for four weeks after treatment, direct sunlight should be avoided as a photosensitive drug is used. In addition, the photosensitive drug administered intravenously can cause accumulation in the kidneys.

Accordingly, given current treatment methods and techniques, at least some of the difficulties/limitations described above need to be addressed:

The invention that is subject of the patent numbered “CN104367349A” in state of the art relates to medical devices and, more specifically, the radiofrequency ablation and automatic biopsy system developed with vacuum elements. Radiofrequency ablation vacuum automatic biopsy system includes elements such as negative pressure automatic biopsy needle, radiofrequency ablation needle, syringe and air suction tube, sharp blade, pressure sensor, and sampling port. Moreover, the system includes a radiofrequency ablation vacuum automatic biopsy gun and a vacuum pump connected to a suction tube. The radiofrequency ablation vacuum automatic biopsy system aims to minimize the problems such as bleeding and tumor implantation in the surrounding tissues during the biopsy procedure and obtain the pathological tissue without contamination by bacteria. In addition, it aims to make the biopsy process simple, fast, and effective by making radiofrequency applications more effective. Thus, it reduces the workload of health personnel.

However, the system in question uses only radiofrequency ablation, and the photothermal ablation technique is not suitable due to the lack of optical infrastructure. Especially during ablation, there is no effective scanning process between 0° and 360°, and the ablation area is not controlled according to the location of the lesions by sliding the probe over the surface. Surface scanning with a micro-electro-mechanical system (MEMS) mirror cannot be combined with thermal ablation therapy and optical imaging procedures. Surface scanning with a micro-electro-mechanical system (MEMS) mirror cannot be combined in any way with thermal ablation therapy and optical imaging procedures. Also, thermal ablation technique and optical imaging cannot be combined in the same endoscopy probe.

The invention that is subject of the patent number “KR101045768B1” in state of the art relates to endoscopic applications and, more specifically, endoscopic mucosal resection. The device consists of nested tubes that can be used with conventional endoscopes, tissue cutting elements, lateral opening, and plates that prevent the advancement of the tissue pulled with negative pressure. The device in question aims to address the limitations encountered with the conventional endoscope during the endoscopic mucosal resection procedure. Examples of limitations of conventional endoscopes include the application of negative pressure perpendicular to the tissue surface, the complex control of the flexible electrosurgical wire used for mucosal resection, and thus possible damage to deep tissue layers and organ perforation. In addition, radiofrequency ablation is targeted as a supportive method to prevent possible bleeding after mucosal resection in the device in question. However, the device in question does not have an optical infrastructure for photothermal ablation. Ablation area control provided by effective scanning between 0°-360° is also not available. In addition, photothermal ablation, radiofrequency ablation, and optical imaging processes cannot be applied together by scanning the tissue surface with a micro-electronic mechanical system (MEMS) mirror-like scanning system.

The invention that is subject of the patent numbered “US2014296742A1” in state of the art relates to medical devices and, more specifically, endoscopic abrasion devices and the method used in the treatment of esophageal diseases. The device in question aims to solve the problems encountered in endoscopic treatment methods by developing an abrasive device that combines an inflatable and rotatable balloon with one or more abrasive elements and a catheter. Examples of these problems are that electrical current and other energy types cause side effects after ablation, circular resection cannot be performed due to organ narrowing, and the treatment of lesions that spread over a large area takes a long time. It also facilitates sample collection by pulling tissue pieces into the catheter groove with negative pressure. However, an effective and adjustable circumferential scanning between 0° and 360° cannot be performed with the device in question. Besides, in the case of a longitudinally extensive lesion, it is not possible to slide the endoscope probe over the mucosal surface during treatment, which shortens the treatment time. In addition, it also lacks a scanning mirror (e.g., micro-electronic mechanical system mirror) and fiber optic components (e.g., GRIN lens). Thus, thermal ablation techniques and optical imaging such as optical tomography cannot be combined in the same endoscope.

The invention that is subject of the patent numbered “US2020016301” in state of the art aims to develop devices and methods for healing the anastomotic wound on the endoluminal surface with negative pressure after the procedure performed on the lumen tissues. Some of the problems encountered during the treatment of colorectal cancer related to luminal organs can be listed as follows; inability to heal the wound in a short time after the surgical intervention, the need for another surgical intervention after colostomy and ileostomy procedures as a temporary solution to stool drainage, the possibility of leakage and abscess at the intestinal junction after resection. Using a drainage pipe, the device in question prevents the leakage of body fluids accumulated at the joint into the body. In addition, it removes body fluids absorbed from the wound surface with a drainage tube passing through the center of the porous absorbent material used. However, the device in question does not have the feature of applying any thermal ablation technique.

There is no ablation area control regarding the location of the lesions, especially with the presented effective scanning between 0°-360° and sliding the probe. In addition, photothermal ablation, radiofrequency ablation, and optical imaging processes cannot be applied together in the same endoscopy probe.

Considering the challenges and the inadequacy of existing devices and systems mentioned above, new approaches and technologies are needed in the relevant technical field.

### SUMMARY OF INVENTION

The present invention discloses an apparatus, system, and method for providing optical (400 nm-2000 nm) imaging and well-confined ablation of abnormal mucosal tissue using negative pressure during endoscopic interventions.

Specifically, the present invention relates to endoscopy devices and systems using optical components (e.g., single-mode and multi-mode fibers, optical fiber with a polished ball-lensed tip, fiber-coupled GRIN lens, coated edge mirrors, scanning mirrors), a stepper motor, and laser and radiofrequency sources to overcome the aforementioned drawbacks and bring new advantages to the relevant technical field.

The advantages and innovations of the present invention over state of the art can be summarized as follows: the invention take advantage of the flexibility of the lining tissue (such as the mucosal tissue layer) and provide well-confined therapeutic depth (i.e., accurate ablation margins) by vacuuming (negative pressure) into a slot that separates the targeted layer from the underlying tissue layers. Moreover, its design allows the apparatus to be glided/slid over the tissue surface, providing a well-confined therapeutic effect for widely disseminated lesions in a single session. Exemplary embodiments for photothermal ablation also aim at accurately controlling the treatment depth depending on the optical penetration depth of the laser beam in the mucosal tissue and the laser energy density at the infrared wavelength range.

In one of the exemplary embodiments, an effective angle-controlled photothermal ablation therapy is also produced by transmitting the laser beam with the rotary scanning mirror of the device into the recessed channel (slot) at any angle from 0° to 360° (in 0.1° steps). In another embodiment, the system consists of photothermal ablation to optical (400 nm-2000 nm) imaging (for example, optical coherence tomography) techniques for both diagnostic and therapeutic (see-and-treat) applications in a single endoscopic apparatus. In one embodiment, the system, including an apparatus, which can be mounted at the distal end of the insertion tube of a conventional endoscopy device, provides both radiofrequency ablation and white light imaging to be performed simultaneously. In one embodiment, the system includes an apparatus that can be mounted at the distal end of the insertion tube of a conventional endoscope, allowing simultaneous radiofrequency ablation and white light imaging. In one embodiment, the system includes an apparatus that can be mounted at the distal end of the insertion tube of a conventional endoscope, allowing simultaneous radiofrequency ablation and white light imaging. Thus, diagnostic and therapeutic features are combined in a single endoscopic device.

### BRIEF DESCRIPTION OF DRAWINGS

The following are descriptions of the accompanying figures showing illustrative embodiments of the present disclosure that clearly explain the objects, features, and advantages of the invention.

FIG. 1A: Representative drawing of the 360-degree treatment apparatus in which an optical waveguide array surrounds the recessed channel into which the target tissue is sucked via negative pressure.

FIG. 1B: Representative drawing of the pieces that make up the casing of the 360-degree treatment apparatus using an optical waveguide array.

FIG. 1C: Representative drawings of the optical components of the 360-degree treatment apparatus using an optical waveguide array.

FIG. 2A: Representative zoom-in drawings of a flat-ended optical fiber and a tapered optical fiber used in the 360-degree treatment apparatus.

FIG. 2B: Representative cross-sectional drawing of the 360-degree treatment apparatus using an optical waveguide array.

FIG. 3A: Representative drawing of the 90-degree treatment apparatus using optical waveguides.

FIG. 3B: Representative drawing of the pieces that make up the casing of the 90-degree treatment apparatus using optical waveguides.

FIG. 4A: Representative zoom-in drawings of a flat-ended optical fiber and a tapered optical fiber used in the 90-degree treatment apparatus.

FIG. 4B: Representative cross-sectional drawing of the 90-degree treatment apparatus using optical waveguides.

FIG. 5A: Representative drawing of the 90-degree treatment apparatus design using an array of optical waveguides coupled with polished ball-lensed tips.

FIG. 5B: Representative drawing of the pieces that make up the casing of the 90-degree treatment apparatus design using an array of optical waveguides coupled with polished ball-lensed tips.

FIG. 5C: Representative drawings of the optical components of the 90-degree treatment apparatus design using an array of optical waveguides coupled with polished ball-lensed tips.

FIG. 6A: Representative zoom-in drawings of optical fibers with polished ball lens ends used in the 90-degree treatment apparatus.

FIG. 6B: Representative cross-sectional drawing of the 90-degree treatment apparatus design using an array of optical waveguides coupled with polished ball-lensed tips.

FIG. 7: Representative drawing of a system using the apparatus embodiments of the disclosure providing circumferential and centered 90-degree/360-degree photo-thermal treatment via the described optical waveguides.

FIG. 8A: Representative drawing of the 360-degree treatment apparatus with a centered conical mirror.

FIG. 8B: Representative drawing of the pieces that make up the casing of the 360-degree treatment apparatus with a centered conical mirror.

FIG. 8C: Representative drawings of the optical components of the 360-degree treatment apparatus with a centered conical mirror.

FIG. 9: Representative cross-sectional drawing of the 360-degree treatment apparatus with a centered conical mirror.

FIG. 10: Representative drawing of a system using the 360-degree treatment apparatus with a centered conical mirror.

FIG. 11A: Representative drawing of the 0 to 360-degree active angle control treatment apparatus.

FIG. 11B: Representative drawing of the pieces that make up the 0 to 360-degree active angle control treatment apparatus' casing.

FIG. 11C: Representative drawings of the optical and mechanical components of the 0 to 360-degree active angle control treatment apparatus.

FIG. 12: Representative cross-sectional drawing of the 0 to 360-degree active angle control treatment apparatus.

FIG. 13: Representative drawing of a system using the 0 to 360-degree active angle control treatment apparatus.

FIG. 14A: Representative drawing of the 0 to 360-degree active angle control treatment and imaging apparatus.

FIG. 14B: Representative drawing of the pieces that make up the 0 to 360-degree active angle control treatment and imaging apparatus.

FIG. 14C: Representative drawings of the optical and mechanical components of the 0 to 360-degree active angle control treatment and imaging apparatus.

FIG. 15: Representative cross-sectional drawing of the 0 to 360-degree active angle control treatment and imaging apparatus.

FIG. 16: Representative drawing of a system using the 0 to 360-degree active angle control treatment and imaging apparatus.

FIG. 17A: Representative drawing of the microelectronic mechanical system (MEMS) mirror based 0 to 90-degree active angle control treatment and imaging apparatus.

FIG. 17B: Representative drawing of the pieces that make up the microelectronic mechanical system (MEMS) mirror based 0 to 90-degree active angle control treatment and imaging apparatus.

FIG. 17C: Representative drawings of the optical and mechanical components of the microelectronic mechanical system (MEMS) mirror based 0 to 90-degree active angle control treatment and imaging apparatus.

FIG. 18A: Representative side drawing of the microelectronic mechanical system (MEMS) mirror based 0 to 90-degree active angle control treatment and imaging apparatus.

FIG. 18B: Representative cross-sectional drawing of the microelectronic mechanical system (MEMS) mirror based 0 to 90-degree active angle control treatment and imaging apparatus.

FIG. 19: Representative drawing of a system using the microelectronic mechanical system (MEMS) mirror based 0 to 90-degree active angle control treatment and imaging apparatus.

FIG. 20A: Representative drawings of the radiofrequency-based 360-degree treatment and optical imaging apparatus.

FIG. 20B: Representative drawing of the radiofrequency-based 360-degree treatment and optical imaging apparatus.

FIG. 20C: Representative drawings of the electrical components of the radiofrequency-based 360-degree treatment and optical imaging apparatus.

FIG. 21A: Representative zoom-in drawings of electrodes used in the radiofrequency-based 360-degree treatment and optical imaging apparatus.

FIG. 21B: Representative cross-sectional drawing of the radiofrequency-based 360-degree treatment and optical imaging apparatus.

FIG. 22A: Representative zoom-in drawings of electrodes used in the radiofrequency-based 90-degree treatment and optical imaging apparatus.

FIG. 22B: Representative drawing of the pieces that make up the radiofrequency-based 90-degree treatment and optical imaging apparatus.

FIG. 22C: Representative zoom-in drawings of electrodes used in the radiofrequency-based 90-degree treatment and optical imaging apparatus.

FIG. 23: Representative cross-sectional drawing of the radiofrequency-based 90-degree treatment and optical imaging apparatus.

FIG. 24: Representative drawings of radiofrequency-based 90-degree/360-degree treatment and optical imaging apparatuses combined with a conventional endoscope tip.

FIG. 25: Representative drawing of a system using the radiofrequency-based 90-degree/360-degree treatment and optical imaging apparatuses.

FIG. 26A: Representative drawing of the 90-degree therapeutic and optical imaging apparatus using radiofrequency ablation and a MEMS mirror.

FIG. 26B: Representative drawing of the pieces that make up the 90-degree therapeutic and optical imaging apparatus using radiofrequency ablation and a MEMS mirror.

FIG. 26C: Representative drawings of the optical, mechanical, and electrical components of the 90-degree therapeutic and optical imaging apparatus using radiofrequency ablation and a MEMS mirror.

FIG. 27A: Representative side drawing of the 90-degree therapeutic and optical imaging apparatus using radiofrequency ablation and a MEMS mirror.

FIG. 27B: Representative cross-sectional drawing of the 90-degree therapeutic and optical imaging apparatus using radiofrequency ablation and a MEMS.

FIG. 28: Representative drawing of a system using the 90-degree therapeutic and optical imaging apparatus using radiofrequency ablation and a MEMS.

### DESCRIPTION OF EMBODIMENTS

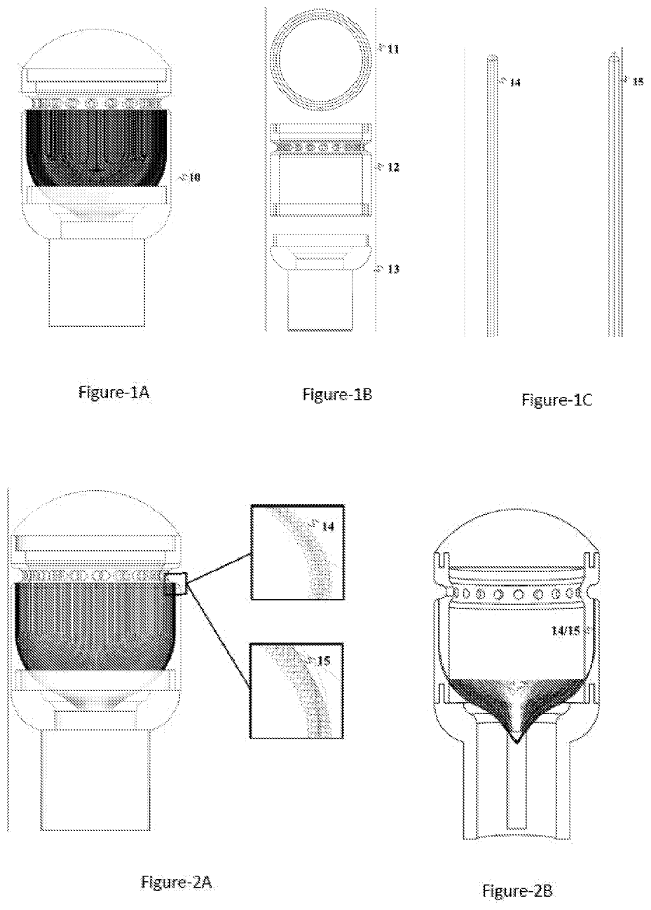

FIG. 1A presents an exemplary embodiment of the 360-degree therapeutic apparatus 10 in which an optical waveguide array surrounds the recessed channel into which the target tissue will be absorbed by negative pressure. The optical waveguide array provides direct transmission of light energy into the recessed channel (slot). Commonly used single-mode or multimode optical fibers are examples of optical waveguides. As shown in FIG. 1B, the apparatus consists of three parts: the top cover 11, a body with a recessed channel 12, and the lower cover 13

The apparatus may include any of the optical components exemplified in FIG. 1C. Essentially a point laser light source, these components can be a flat-ended optical fiber 14 or a tapered optical fiber 15. On the other hand, the tapered optical fiber 15 ensures that the beam is tightly focused on the target tissue. Thus, the tapered optical fiber 15 can be used in ablation processes using relatively low-power laser sources.

The top cover 11 forms the top of the probe. The lower cover 13 forms the bottom of the apparatus. The top cover 11 and the lower cover 13, whose body overlaps the recessed channel, form the entire casing of the 360-degree therapeutic apparatus 10 in which an optical waveguide array surrounds the recessed channel. The outer diameter of the apparatus may be, but are not limited to, between 10 mm and 20 mm for appropriate intervention in the human gastrointestinal tract. The recessed channel (i.e., the slot) has a circumferential row of holes to provide vacuum conduction and creates a physical boundary to the vacuum-retracted tissue. In other words, the tissue is kept in a particular volume, and this limited volume, well-confined volume, is treated. All parts are made of biocompatible material that does not react with biological tissues and does not cause adverse side effects (e.g., infection). The biocompatible material used in the embodiments of the invention has a high surface hardness, low water absorption, dimensional stability, and workable form that is resistant to scratches, chemicals, ultraviolet rays, and atmospheric conditions. In addition, the biocompatible material, particularly for the body with a recessed channel 12, is optically transparent and has a high (>90%) light transmittance, especially in the spectral range of 400 nm-2000 nm. Examples of biocompatible materials are polymethyl methacrylate (PMMA) or polycarbonate (PC).

As shown in FIG. 2A, the optical components (a flat-ended optical fiber 14 or a tapered optical fiber 15) exemplified in FIG. 1C are arranged circumferentially at the bottom of the slot, nesting in the slot of the body with a recessed channel 12. The working principle of the exemplary embodiment apparatus, whose cross-sectional view is presented in FIG. 2B, is summarized as follows: An optical waveguide array surrounding the recessed channel transmits the 360-degree therapeutic laser energy. The target mucosa tissue gently pulled into the slot with negative pressure is exposed to circumferential thermal damage based on photo-thermal coagulation by directly encountering laser energy transmitted by circumferentially arrayed optical waveguides at the base of the channel.

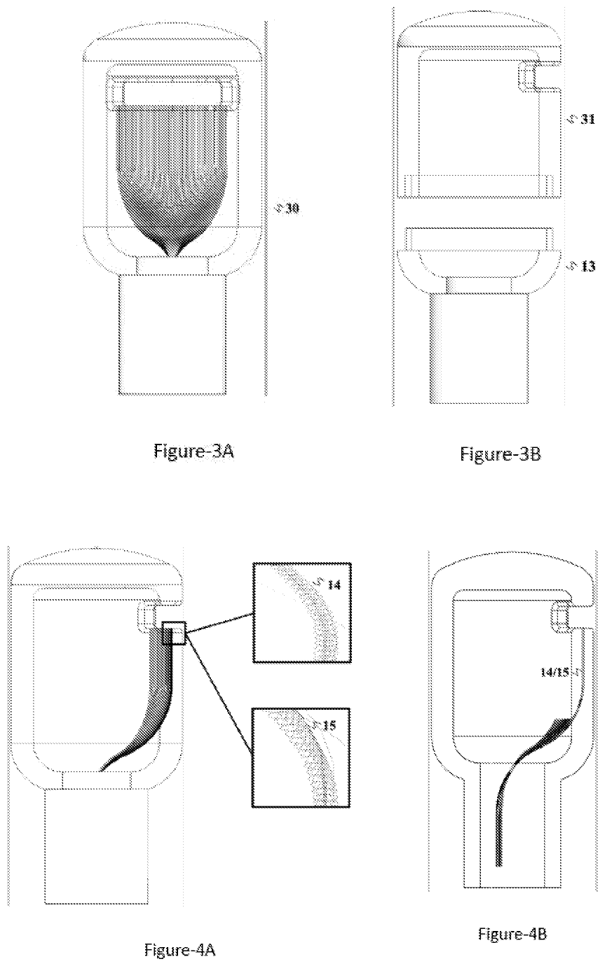

FIG. 3A demonstrates a representative embodiment of the present disclosure for the 90-degree therapeutic apparatus using optical waveguides 30. As shown in FIG. 3B, the apparatus consists of two parts: the lower cover 13 and the upper body with a 90° opening 31. The upper body with a 90-degree opening 31 includes the recessed channel of the apparatus. The upper body with a 90-degree opening 31 and the lower cover 13 form the whole of the 90-degree treatment apparatus using optical waveguides 30. All parts are made of biocompatible material like other arrangements.

As shown in FIG. 4A, optical waveguides are located at the base of the arc-shaped enclosure at a 90-degree angle to the upper body. A flat-ended optical fiber 14 or a tapered optical fiber 15 exemplified in FIG. 4A can transmit laser energy to the slot.

The working principle of the 90° treatment apparatus, whose cross-sectional view is presented in FIG. 4B, is similar to the working principle of the 360-degree therapeutic apparatus 10 described in FIG. 2B. The target tissue layer gently pulled to the 90-degree opening in the upper body with negative pressure is locally exposed to well-confined thermal damage based on photothermal tissue coagulation.

In another exemplary embodiment of the present disclosure, FIG. 5A presents a 90-degree therapeutic apparatus design using an array of optical fibers coupled with polished ball-lensed tips 55. As shown in FIG. 5B, the apparatus consists of four parts: a d-shaped cylinder to fix the optical waveguides in place 51, a glass ferrule 52, a glass top cover with grooves 53, and a lower body with a 90-degree opening 54.

The apparatus may comprise optical fibers (single-mode or multimode) coupled with polished ball-lensed tips 55 for delivering laser energy to the target tissue, as exemplified in FIG. 5C.

The glass top cover with grooves 53 forms the top of the apparatus while aligning the ferrule to the center. The glass ferrule 52 becomes a protective and enveloping sheath by placing over the d-shaped cylinder to fix the optical waveguides in place 51. In addition, the glass top cover with grooves 53 overlaps with the lower body with a 90-degree opening 54 and forms the whole of the apparatus. The laser beam reflected from the polished surface of the ball-lensed tip is side-fired for therapeutic purposes (i.e., photothermal coagulation or photothermal ablation) and directed perpendicular to the tissue.

Like other embodiments, all parts constituting the apparatus are biocompatible materials. Besides, the biocompatible material used in the lower body with a 90-degree opening 54 has high optical transmission.

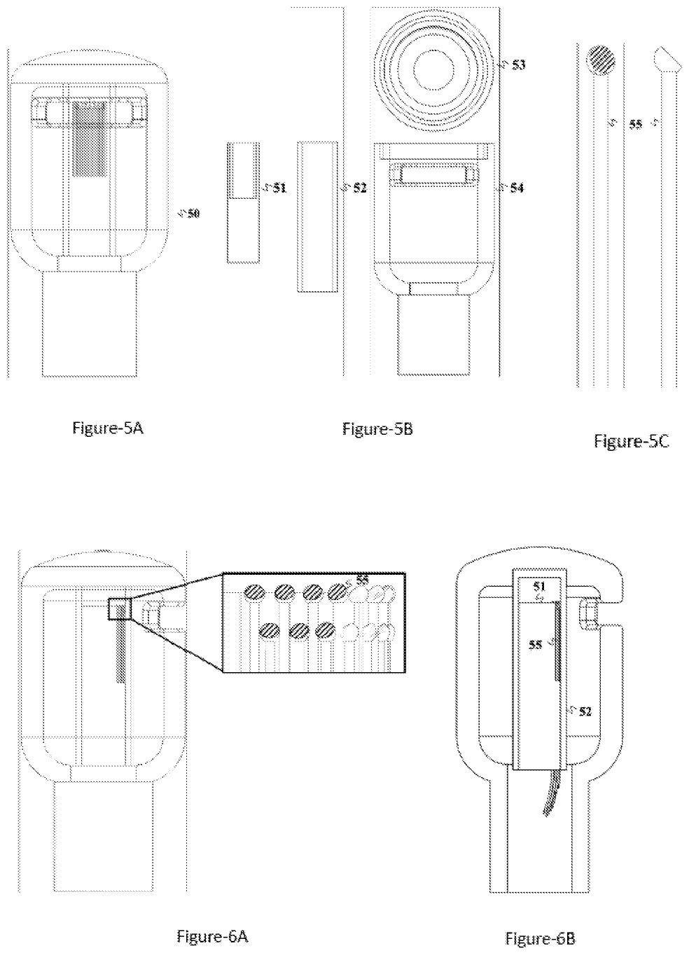

As shown in FIG. 6A, optical fibers with polished ball lens ends can be arranged in an arc to form a double-row, an upper and a lower one, respectively, at an aperture of 90-degree.

The working principle of the exemplary embodiment apparatus, whose cross-sectional view is presented in FIG. 6B, is summarized as follows: the target tissue layer is gently pulled with negative pressure into the 90° opening of the apparatus. Laser energy is side-fired through the glass ferrule 52 to the recessed opening via the optical fibers coupled with polished ball-lensed tips 55 arranged in double rows. Thus, the target tissue is exposed to well-confined therapeutic thermal damage based on the photothermal effect (coagulation or ablation). The double-row fiber-optic array provides a photothermal effect on the large surface of the tissue drawn into the opening of the apparatus.

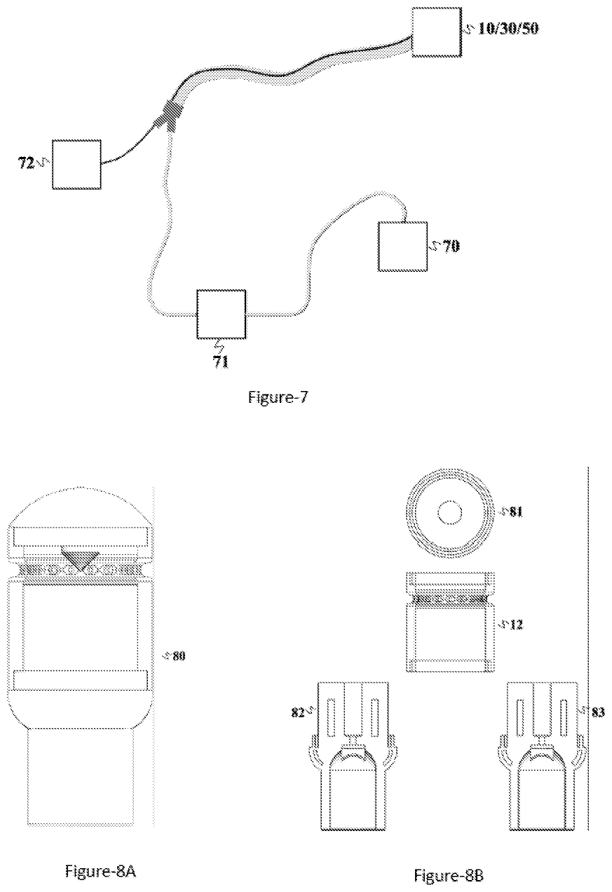

An exemplary arrangement of a system using the apparatus embodiments of the disclosure providing circumferential and centered 90-degree/360-degree photo-thermal treatment via the described optical waveguides is shown in FIG. 7. The system includes an electric vacuum pump 70, a manometer 71, a light source 72, and a 90-degree/360-degree therapeutic apparatus 10/30/50. The apparatus embodiments described in FIG. 1A, FIG. 3A, and FIG. 5A can be used as a therapy probe in the system. The electric vacuum pump 70 creates the negative pressure, i.e., vacuum, necessary to pull the target tissue layers into the recessed channel of the apparatus. The negative pressure applied to the tissue can be in the range of −150 mmHg to −760 mmHg. A manometer 71 is connected in series to the electric vacuum pump 70 to measure the negative pressure value continuously and directly. A light source 72 that radiates in the near-infrared spectrum is used to expose the target tissue to thermal damage, i.e., photo-thermal ablation of the mucosa tissue in the recessed channel. At the near-infrared wavelength, the treatment depth is controlled depending on the optical penetration depth of the laser beam in the mucosal tissue and the laser irradiance for the fixed spot size. Therefore, another feature that provides a limited depth of treatment is the laser beam with a near-infrared wavelength. The light source includes, but is not limited to, an amplified spontaneous laser source, semiconductor laser, fiber laser, solid-state laser. The light source 72 may be an optical waveguide coupled to efficiently transmit the laser beam to the apparatus. The laser beams may be a fiber optic bundle containing multiple optical waveguides rather than a single optical waveguide.

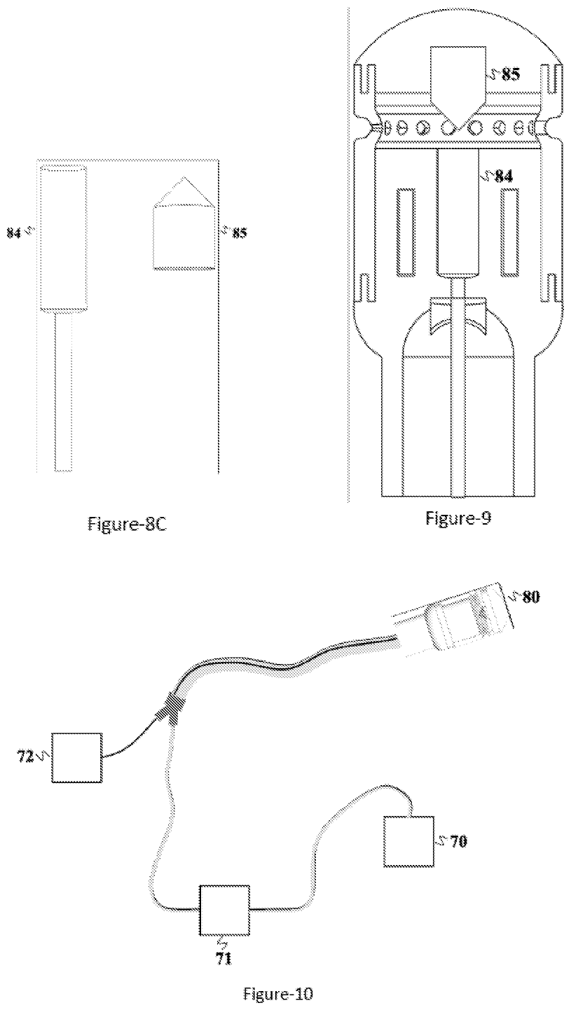

According to the conical mirror-centered arrangement of the invention, FIG. 8A presents an exemplary application of the 360-degree therapeutic apparatus with a centered conical mirror 80. As shown in FIG. 8B, the apparatus consists of four parts: a top cover for 360-degree reflection 81, a body with a recessed channel 12, a lower-left cover for a single beam 82, and a lower-right cover for a single beam 83.

The treatment apparatus includes optical components exemplified in FIG. 8C. These optical components are an optical waveguide coupled collimator 84 and a conical mirror 85. The optical waveguide coupled collimator 84 includes, but is not limited to, fiber collimators using a Graded-Index (GRIN) lens or fiber-coupled aspherical lens collimators. In the invention's preferred embodiment, the conical mirror 85 may have a gold, silver, or aluminum coating (>80% reflectance). In addition, the reflective surface of the conical mirror 85 may have a protective coating against scratching and oxidation. Silicon monoxide (SiO) or silicon dioxide (SiO2) can be used as a protective coating.

The top cover for 360-degree reflection 81 forms the top of the apparatus. The conical mirror 85, fixed in place on the top cover and positioned in the center, reflects the laser beam 360 degrees.

The lower-left cover for a single beam and the lower-right cover for a single beam assemble the bottom of the apparatus in which the optical waveguide coupled collimator 84 can be fixed in place. The optical waveguide coupled collimator 84 transmits the therapeutic laser beam to the mirror centered in the apparatus.

The top and lower covers overlapping the body with a recessed channel 12 completed the apparatus. All parts of the apparatus are manufactured from biocompatible materials, similar to other embodiments.

The working principle of the exemplary embodiment apparatus, whose cross-sectional view is presented in FIG. 9, is summarized as follows: The optical waveguide coupled collimator transmits the laser beam to the apparatus. The collimated laser beam is reflected, 360-degree, circumferentially by the conical mirror 85 fixed on the top cover of the apparatus. The circular plane formed by the beam reflected from the conical surface is explicitly positioned by targeting the recessed channel of the apparatus. Thus, the target mucosal tissue, slowly pulled into the recessed channel by negative pressure, is circumferentially exposed to thermal damage based on photo-thermal tissue coagulation.

An exemplary arrangement of a system utilizing the apparatus embodiments of the disclosure providing circumferential, 360-degree, photo-thermal treatment via a centered conical mirror is shown in FIG. 10. The system includes an electric vacuum pump 70, a manometer 71, a light source 72, and the 360-degree therapeutic apparatus with a centered conical mirror 80. Unlike the system described in FIG. 7, the laser beam is transmitted to the treatment apparatus by a single optical waveguide instead of an optical waveguide beam. The reflection of the laser beam aligned directly to the conical mirror 85 surfaces by scattering in a 360-degree circular plane affects the energy density per unit area. Therefore, a high-power laser beam from the light source 72 may be required to induce photo-thermal coagulation of the target mucosa tissue, which is gently sucked into the recessed channel of a 360-degree therapeutic apparatus with a centered conical mirror 80.

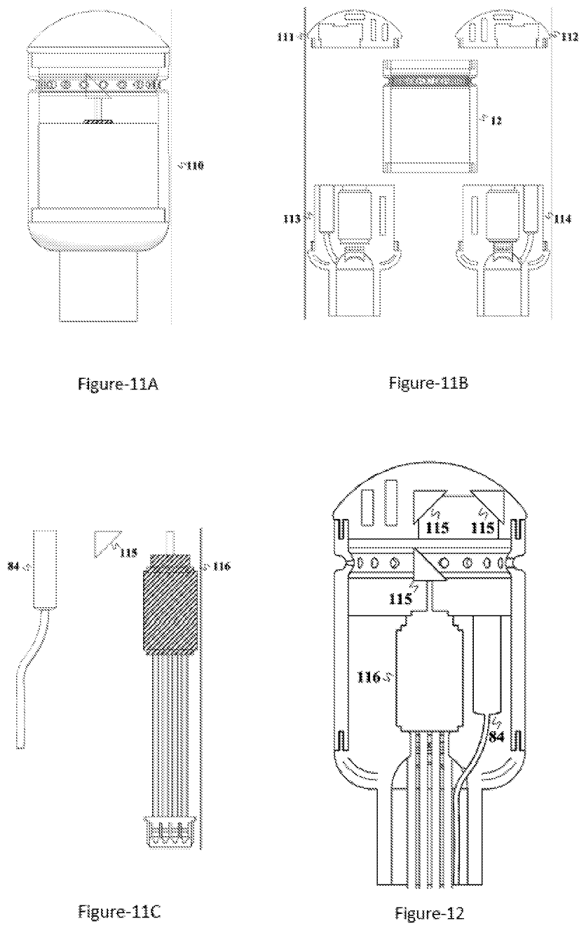

According to the arrangement of the present disclosure providing active angle control, FIG. 11A presents an exemplary embodiment of a 0 to 360-degree active angle control therapeutic apparatus 110. As shown in FIG. 11B, the apparatus consists of five parts: an upper left cover for single-beam reflection 111, an upper right cover for single-beam reflection 112, a body with a recessed channel 12, a lower left cover for single-beam transmission and scanning 113, and a lower right cover for single-beam transmission and scanning 114.

The treatment apparatus includes optical and mechanical components exemplified in FIG. 11C. These components are an optical waveguide coupled collimator 84, right-angle mirrors 115 with similar specifications, and a stepper motor 116. Three, right-angle mirrors 115 with similar technical characteristics are used. The right-angle mirror 115 may have a gold, silver, or aluminum coating (>80% reflectance). In addition, the reflective surface of the right-angle mirror 115 may have a protective coating layer against scratching and oxidation. The stepper motor 116 has high stepping accuracy. Therefore, the step resolution can be <0.1°. The voltage and load values applied to the stepper motor 116 determine the highest possible rotation speed of the motor. The stepper motor 116 can be actively controlled by clockwise and counterclockwise rotational motion. The upper left cover for single-beam reflection 111 and the upper right cover for single-beam reflection 112 form the top of the apparatus.

The caps overlap to assemble a top cover with mirror slots to place the two right-angle mirrors 115. The right-angle mirrors 115 vertically reflect the laser beam transmitted to the apparatus onto the shaft rod of the stepper motor 116.

The lower left cover for single-beam transmission and scanning 113 and the lower right cover for single-beam transmission and scanning 114 form the bottom of the apparatus. The covers overlap to assemble a bottom cover with slots to place the optical waveguide coupled collimator 84 and the stepper motor 116. The optical waveguide coupled collimator 84 transmits and collimated the therapeutic laser beam to the apparatus. The stepper motor 116 is used for the circumferential or local surface scanning laser beam.

The top parts and bottom parts overlapping the body with a recessed channel 12 complete the case of the apparatus. All parts are manufactured from a biocompatible and optically transparent material like other embodiments.

The working principle of the exemplary embodiment apparatus, whose cross-sectional view is presented in FIG. 12, is summarized as follows: the optical waveguide coupled collimator 84 transmits the laser beam to the apparatus.

Two right-angle mirrors 115 fixed in place on the top cover of the apparatus reflect the collimated laser beam to another right-angle mirror 115 affixed to the distal tip of the shaft rod of the stepper motor 116. Circumferential side-firing of the laser beam is achieved by the rotation of the stepper motor 116. With the control of the analog or digital electrical signal driving the motor, scanning can be performed in circumferential (360°) or between specified angles. Thus, the target mucosal tissue gently pulled into the recessed canal with negative pressure is exposed to photo-thermal coagulation.

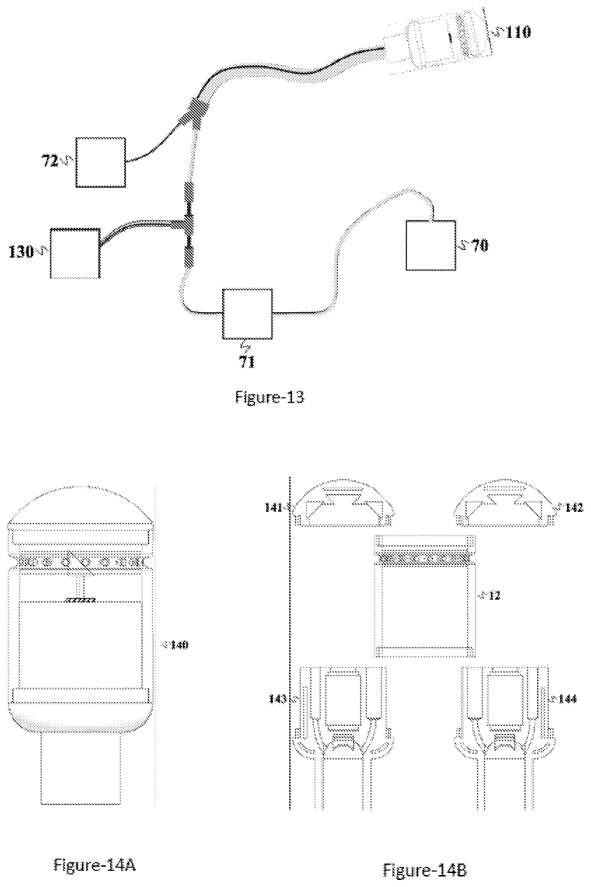

An exemplary arrangement of a system utilizing the apparatus embodiments of the present disclosure that provides photo-thermal therapy with active angle control from 0 to 360 degrees is shown in FIG. 13. The system includes an electric vacuum pump 70, a manometer 71, a light source 72, a driver board 130, and a 0 to 360-degree active angle control therapeutic apparatus 110. Unlike the system described in FIG. 10, the driver board 130 drives a stepper motor 116 that enables the laser beam to scan the surface.

The biocompatible material used in the 360-degree therapeutic apparatus 10, the 90-degree therapeutic apparatus using optical waveguides 30, 90-degree therapeutic apparatus using an array of optical waveguides coupled with polished ball-lensed tips 50, or the conical mirror-centered 360-degree therapeutic apparatus 80, or the body with a recessed channel 12 of the 0 to 360-degree active angle control therapeutic apparatus 110, the upper body with a 90-degree opening 31, a lower body with a 90-degree opening 54 may be made of polymethyl methacrylate (PMMA) or polycarbonate (PC).

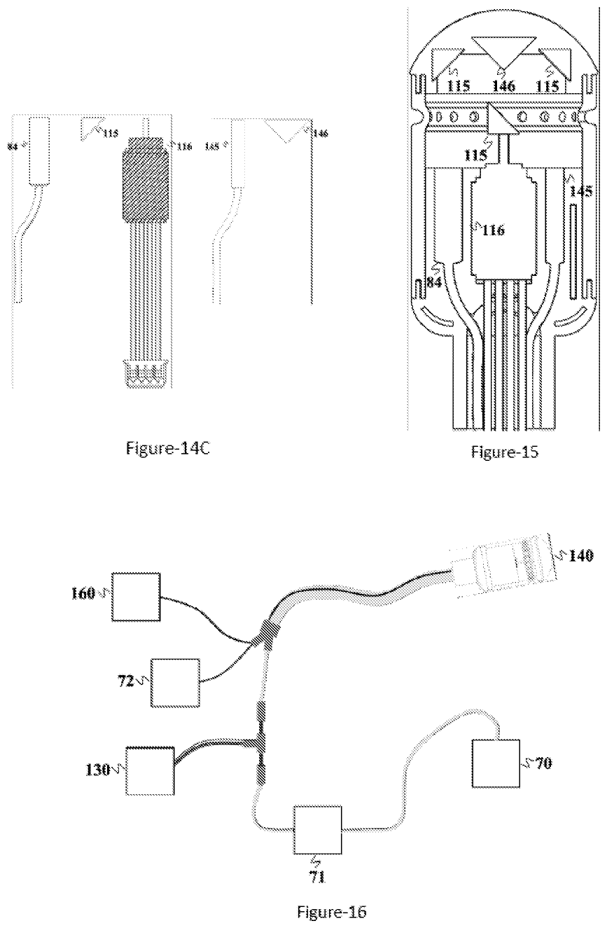

According to another arrangement of the present disclosure that provides active angle control, FIG. 14A presents an exemplary embodiment of a 0 to 360-degree active angle control therapeutic and imaging apparatus 140. As shown in FIG. 14B, the apparatus consists of five parts: an upper left cover for double-beam reflection 141, an upper right cover for double-beam reflection 142, a body with a recessed channel 12, a lower left cover for double-beam transmission and scanning 143, and a lower right cover for double-beam transmission and scanning 144.

As exemplified in FIG. 14C, the apparatus includes the optical waveguide coupled collimator 84, the right-angle mirror 115, the stepper motor 116, the optical waveguide coupled focusing lens 145, and a knife-edge prism mirror 146. In the preferred application of the disclosure, the knife-edge prism mirror 146, which has similar technical features with the right-angle mirror 115, may have a gold, silver, or aluminum coating (>80% reflectance). In addition, the reflective surface of the knife-edge prism mirror 115 may have a protective coating layer against scratching and oxidation.

The optical waveguide coupled focusing lens 145 includes, but is not limited to, a fiber-coupled Graded-Index (GRIN) lens or a fiber-coupled aspherical focusing lens.

The upper left cover for double-beam reflection 141 and the upper right cover for double-beam reflection 142 form the top of the apparatus. The upper left cover for single-beam reflection 111 and the upper right cover for single-beam reflection 112 form the top of the apparatus.

The caps overlap to assemble the top cover with mirror slots to place the right-angle mirrors 115 and a knife-edge prism mirror 146. The right-angle mirrors 115 vertically reflect the laser beam transmitted to the apparatus onto the shaft rod of the stepper motor 116.

The top and bottom parts overlapping the body with a recessed channel 12 set up the whole of the apparatus. As with other embodiments, all parts that make up the casing of the apparatus are made of a biocompatible material.

The working principle of the exemplary embodiment apparatus, whose cross-sectional view is presented in FIG. 15, is summarized as follows: An optical waveguide coupled collimator 84 transmits and collimates the therapeutic laser beam to the apparatus and then aligns the collimated beam on the right-angle mirrors 115, which is fixed in place of the top cover. Similarly, an optical waveguide coupled focusing lens 145 transmits and collimates the therapeutic laser beam to the apparatus and then aligns the collimated beam on another right-angle mirror 115, which is fixed in place of the top cover. Laser beams are reflected from both right-angle mirrors 115 to a knife-edge prism mirror 146, fixed in its slot in the top cover and aligned to another right-angle mirrors 115 affixed to the distal tip of the shaft rod of the stepper motor 116. Surface scanning of the laser beams is achieved by the rotational motion of the stepper motor 116. The scanning beam is explicitly positioned by targeting the recessed channel of the apparatus. Thus, the target mucosal tissue gently pulled into the recessed channel with negative pressure is exposed to photo-thermal coagulation.

An exemplary arrangement of a system utilizing the apparatus embodiments of the present disclosure that provides photo-thermal therapy with active angle control from 0 to 360 degrees is shown in FIG. 16. The system includes an electric vacuum pump 70, a manometer 71, a light source 72, a driver board 130, an optical (400 nm-2000 nm) imaging device 160, and a 0 to 360-degree active angle control therapeutic and imaging apparatus 140. Unlike the system described in FIG. 13, an optical (400 nm-2000 nm) imaging device 160 is used to perform imaging of the target mucosal tissue pulled into the recessed channel, mainly during photo-thermal coagulation. For example, optical coherence tomography (OCT), confocal imaging, conventional white light imaging, or narrowband imaging may be used as optical imaging modalities.

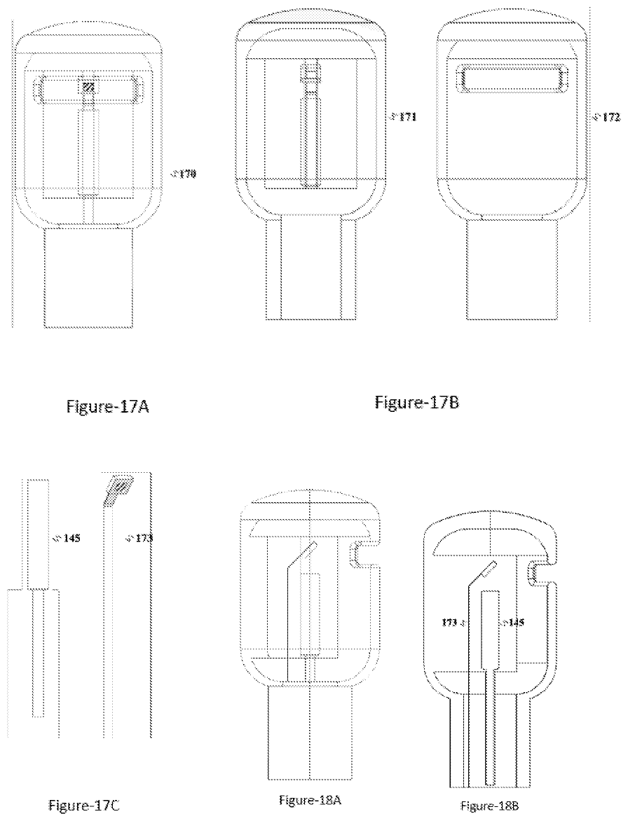

According to another arrangement of the present disclosure, FIG. 17A presents an exemplary embodiment of a MEMS mirror based 0 to 90-degree active angle control therapeutic and imaging apparatus 170. As shown in FIG. 17B, the casing of the probe consists of a total of two pieces: a back cover for single-beam reflection 171 and a front cover with a 90-degree opening 172. The treatment and imaging apparatus includes optical and mechanical components exemplified in FIG. 17C. These components are an optical waveguide coupled focusing lens 145 and a MEMS mirror with a flexible printed circuit 173. In the preferred application of the invention, the MEMS mirror may have a gold, silver, or aluminum coating (>80% reflectance). In addition, the mirror's reflective surface may have a protective coating layer against scratching and oxidation. The back cover for single-beam reflection 171 forms the back of the apparatus. The back cover, fixing the optical waveguide coupled focusing lens 145 and a MEMS mirror with a flexible printed circuit 173, overlaps with a front cover with a 90-degree opening 172 to assemble the therapeutic and imaging apparatus. As with other embodiments, all parts of the apparatus are made of biocompatible and optically transparent material.

As shown in FIG. 18A, the MEMS mirror with a flexible printed circuit 173 is positioned in the direction and above an optical waveguide coupled focusing lens 145. The optical waveguide coupled focusing lens 145 transmits the therapeutic or imaging laser beam to the apparatus and focuses on the MEMS mirror's surface. The MEMS mirror with a flexible printed circuit 173 is used for 0 to 90-degree surface scanning of the transmitted laser beam.

The working principle of the exemplary embodiment apparatus, whose cross-sectional view is presented in FIG. 18B, is summarized as follows: The optical waveguide coupled focusing lens 145 transmits the laser beam to the apparatus. The MEMS mirror with a flexible printed circuit 173 aligned at the top of the optical waveguide coupled focusing lens 145 reflects the focused beam to the apparatus' opening. A micro-electro-mechanical system (MEMS) mirror controlled by a flexible printed circuit enables the mirror to move at certain angles and performs scanning in the range between 0 and 90-degree. Thus, the target mucosal tissue gently pulled into the opening with negative pressure is locally imaged or subjected to thermal injury based on photo-thermal coagulation.

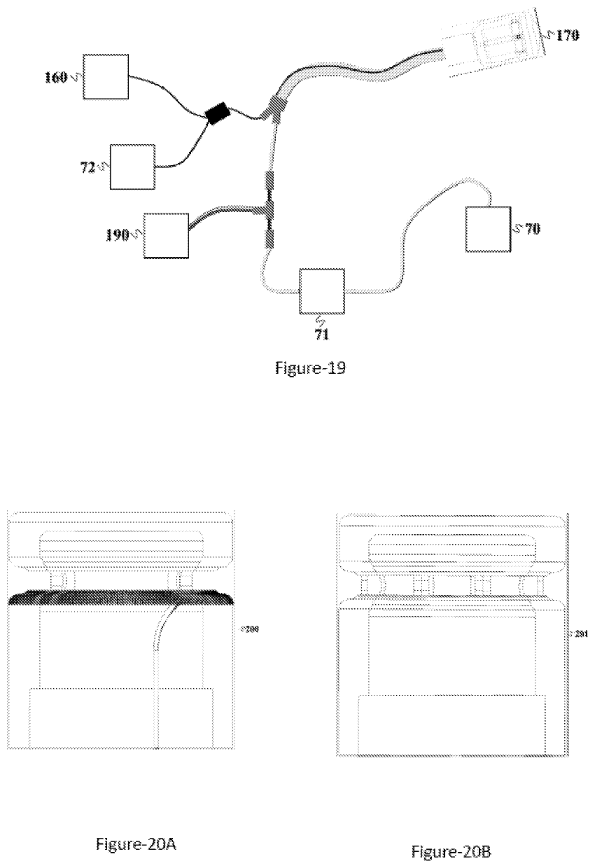

An exemplary arrangement of a system utilizing the apparatus embodiments of the present disclosure that provides 0 to 90-degree active angle control for treatment and imaging based on a micro-electro-mechanical system (MEMS) mirror is shown in FIG. 19.

The system includes an electric vacuum pump 70, a manometer 71, a flexible printed circuit board 190, a light source 72, an optical (400 nm-2000 nm) imaging device 160, and a MEMS mirror based 0 to 90-degree active angle control therapeutic and imaging apparatus 170. Unlike the system described in FIG. 16, instead of using a driver board 130 to drive the stepper motor 116, a flexible printed circuit board 190 is used to control a MEMS mirror for laser surface scanning. A light source 72 is used for photo-thermal coagulation of the target tissue, which is sucked in the opening of the apparatus with negative pressure, and an optical (400 nm-2000 nm) imaging device 160 is employed for imaging.

The biocompatible material used in the listed parts can be polymethyl methacrylate (PMMA) or polycarbonate (PC): a 0 to 360-degree active angle control therapeutic and imaging apparatus 140; the body with a recessed channel 12 of a MEMS mirror based 0 to 90-degree active angle control therapeutic and imaging apparatus 170; and a front cover with a 90-degree opening 172.

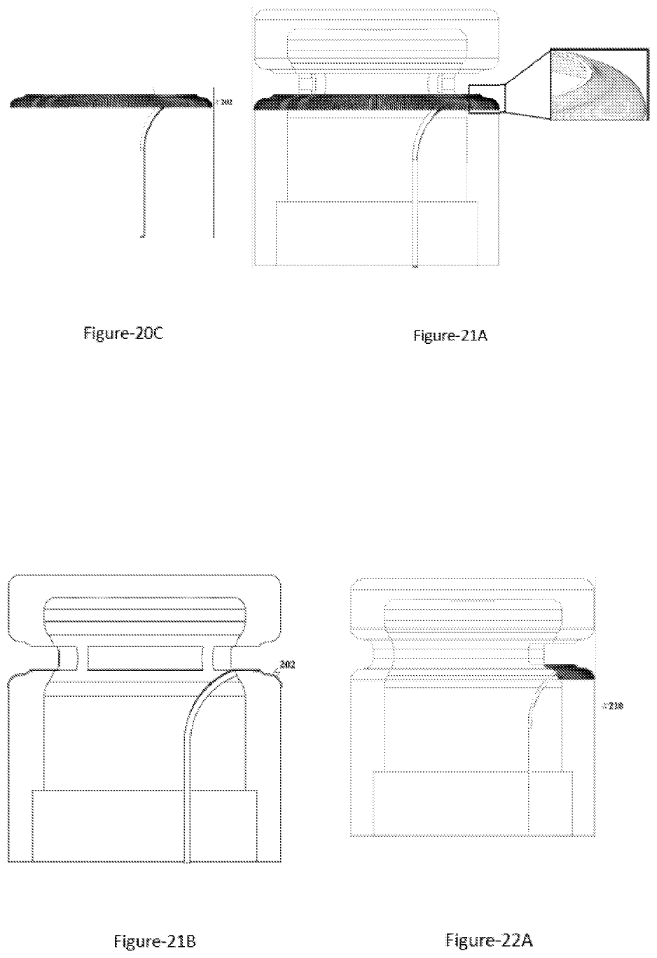

According to another arrangement of the present disclosure, FIG. 20A presents an exemplary embodiment of a radiofrequency-based 360-degree therapeutic and optical imaging apparatus 200. As shown in FIG. 20B, the casing of the apparatus consists of a single piece, that is, a body with a 360-degree opening 201.

The apparatus contains a 360-degree electrode array 202, exemplified in FIG. 20C. The electrodes can be made of copper metal. The 360-degree electrode array 202 is in a flexible form to be placed over the opening of the apparatus.

Electrodes transmit the alternating current for thermal therapeutic purposes. The alternating current frequency can be in the range of 450-500 kHz.

As shown in FIG. 21A, the electrodes exemplified in FIG. 20C are placed in the body with a 360-degree opening 201. The apparatus is made of an optically transparent and, at the same time, biocompatible material.

The working principle of the exemplary embodiment apparatus, whose cross-sectional view is presented in FIG. 21B, is summarized as follows: Electrodes transmit the alternating current at the radiofrequency energy level to the apparatus. Thus, the target mucosal tissue gently pulled into the opening with negative pressure is fully optically imaged and subjected to thermal injury based on radiofrequency ablation.

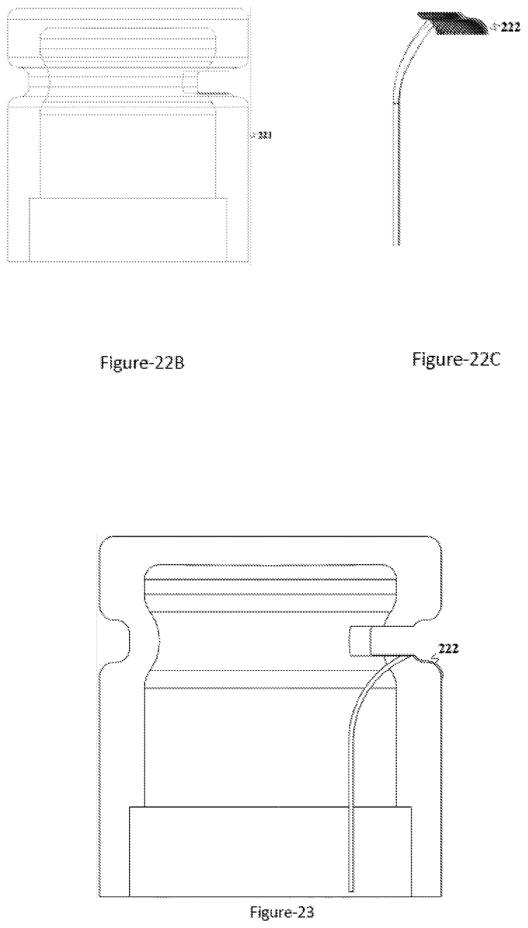

According to another arrangement of the present disclosure, FIG. 22A presents an exemplary embodiment of a radiofrequency-based 90-degree therapeutic and optical imaging apparatus 220. As shown in FIG. 22B, the casing of the apparatus consists of a single piece, that is, a body with a 90-degree opening 221.

The apparatus contains a 90-degree electrode array 222, exemplified in FIG. 22C. The electrodes can be made of copper metal. The 90-degree electrode array 222 is in a flexible form to be placed over the opening of the apparatus.

The apparatus is made of an optically transparent and, at the same time, biocompatible material.

The working principle of the apparatus, whose exemplary cross-sectional view is given in FIG. 23, is the same as that described in FIG. 21B.

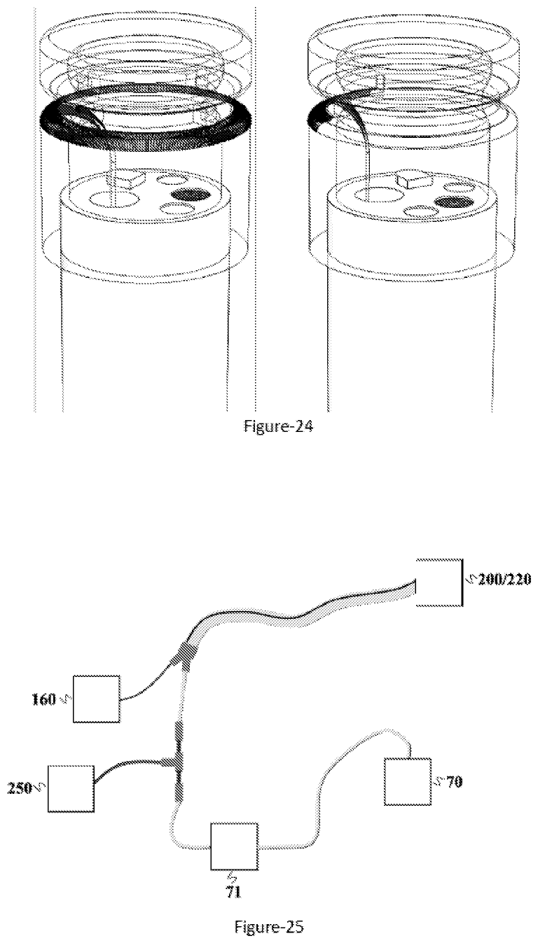

As shown in FIG. 24, radiofrequency-based 90-degree/360-degree treatment and optical imaging apparatuses can be combined with a conventional endoscope tip. Thus, the optically transparent apparatus takes advantage of the standard imaging features provided by the endoscopy system and performs well-confined radiofrequency ablation.

An exemplary arrangement of a system utilizing the apparatus embodiments of the present disclosure that provides radiofrequency-based 90-degree/360-degree treatment and optical imaging is shown in FIG. 25. The system includes an electric vacuum pump 70, a manometer 71, an optical (400 nm-2000 nm) imaging device 160, a radiofrequency generator 250, and a radiofrequency-based 90-degree/360-degree therapeutic and optical imaging apparatus 200/220.

Unlike the system described in FIG. 7, in this system, a radiofrequency generator 250 is used instead of the light source 72. The radiofrequency generator 250 is used to generate electromagnetic radiation for tissue ablation. The energy produced by the generator is transferred to the electrodes. Well-confined, radiofrequency-induced thermal damage occurs with the direct contact of the electrode array to the target tissue, which is sucked into the recessed channel via negative pressure.

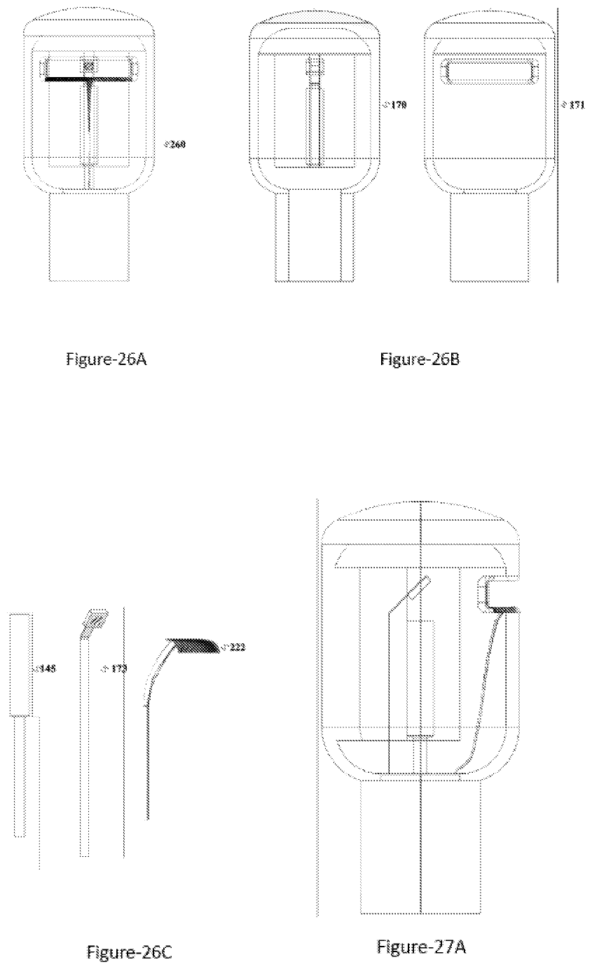

According to another arrangement of the present disclosure, FIG. 26A presents an exemplary embodiment of a 90-degree therapeutic and optical imaging apparatus using the radiofrequency ablation method and a MEMS mirror 260. As shown in FIG. 26B, the casing of the probe consists of a total of two pieces: a back cover for single-beam reflection 171 and a front cover with a 90-degree opening 172.

The treatment and imaging apparatus includes optical and mechanical components exemplified in FIG. 26C. These components are an optical waveguide coupled focusing lens 145, a MEMS mirror with a flexible printed circuit 173, and a 90-degree electrode array 222.

The back cover for single-beam reflection 171 forms the rear of the apparatus. The back cover, fixing the optical waveguide coupled focusing lens 145 and a MEMS mirror with a flexible printed circuit 173, overlaps with a front cover with a 90-degree opening 172 to which the electrode array is affixed, assembling the treatment and imaging apparatus.

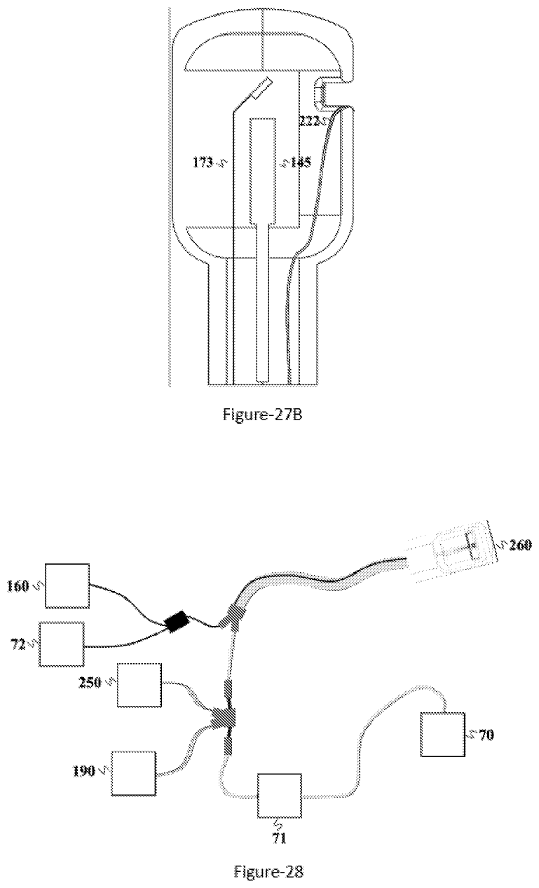

As shown in FIG. 27A, the MEMS mirror with a flexible printed circuit 173 is positioned in the direction and above an optical waveguide coupled focusing lens 145. The optical waveguide coupled focusing lens 145 transmits the therapeutic or imaging laser beam to the apparatus and focuses on the MEMS mirror's surface. The MEMS mirror with a flexible printed circuit 173 is used for 0 to 90-degree surface scanning of the transmitted laser beam. The 90-degree electrode array 222 is used to transmit alternating current for therapeutic purposes induced by radiofrequency ablation.

The working principle of the exemplary embodiment apparatus, whose cross-sectional view is presented in FIG. 27B, is summarized as follows: The optical waveguide coupled focusing lens 145 transmits the laser beam to the apparatus. The MEMS mirror with a flexible printed circuit 173 aligned at the top of the optical waveguide coupled focusing lens 145 reflects the focused beam to the apparatus' opening. A micro-electro-mechanical system (MEMS) mirror controlled by a flexible printed circuit enables the mirror to move at certain angles and performs scanning in the range between 0 and 90-degree. Thus, the target mucosal tissue gently pulled into the opening with negative pressure is locally imaged or subjected to thermal injury based on radiofrequency ablation. Moreover, the target tissue can also be exposed to thermal injury based on photo-thermal ablation by transmitting a therapeutic laser beam via the MEMS mirror with a flexible printed circuit 173.

An exemplary arrangement of a system utilizing the apparatus embodiments of the present disclosure that provides radiofrequency ablation induced thermal treatment or laser-induced photo-thermal treatment and optical imaging is shown in FIG. 28. The system includes an electric vacuum pump 70, a manometer 71, a flexible printed circuit board 190, a radiofrequency generator 250, an optical (400 nm-2000 nm) imaging device 160, a light source 72, and a 90-degree therapeutic and optical imaging apparatus using the radiofrequency ablation method and a MEMS mirror 260. Unlike the system described in FIG. 19, a radiofrequency generator 250 is used in addition to the light source 72 used for therapeutic purposes. The radiofrequency generator 250 is used to generate electromagnetic radiation for tissue ablation. The energy produced by the generator is transferred to the electrodes. Simultaneously, imaging is performed by scanning the surface with the laser beam transmitted to the MEMS mirror. In addition to optical imaging, photo-thermal coagulation can also be performed using a therapeutic light source.

In all of the exemplary embodiments listed in the present disclosure, the apparatus can be controlled manually or automatically; it can be moved by turning or sliding along the mucosal surface as desired. The angle values mentioned in the detailed description of the invention are the angles that the apparatus provides for treatment and imaging when it is in the adjusted position. Some exemplary apparatuses have a fixed application angle of 90 degrees or 360 degrees, while some exemplary apparatuses are designed to be applied at any angle range from 0 to 90 degrees or 0 to 360 degrees. Thus, for situations where the width and length of the mucosal lesion, that is, the area of spread, varies, apparatus embodiments that allow active angle control and the methods of sliding and rotating on the tissue surface are disclosed.

The biocompatible material used in the listed parts can be polymethyl methacrylate (PMMA) or polycarbonate (PC): the radiofrequency-based 360-degree therapeutic and optical imaging apparatus 200; the radiofrequency-based 90-degree therapeutic and optical imaging apparatus 220; the 90-degree therapeutic and optical imaging apparatus using the radiofrequency ablation method and a MEMS mirror 260; a body with a 360-degree opening 201; the body with a 90-degree opening 221; and a front cover with a 90-degree opening 172.

1. A thermal therapy system operating between 0° to 360° to be used in endoscopic medical therapy applications and especially photothermal (laser) ablation/coagulation therapy applications, characterized in that it comprises:
an electric vacuum pump 70 that creates a negative pressure necessary to pull the target tissue into the recessed channel of the therapeutic apparatus,
a manometer 71 that is connected in series to the electric vacuum pump 70 and continuously measures the negative pressure value,
a light source 72 that exposes the target tissue pulled into the recessed channel of the therapeutic apparatus to the photothermal ablation/coagulation,
a 360-degree therapeutic apparatus 10 in which an optical waveguide array surrounds the recessed channel, or a 90-degree therapeutic apparatus using optical waveguides 30, or a 90-degree therapeutic apparatus using an array of optical waveguides coupled with polished ball-lensed tips 50, or a 360-degree therapeutic apparatus with a centered conical mirror 80, or a 0 to 360-degree active angle control therapeutic apparatus 110, with at least one of them being connected to the electric vacuum pump 70, manometer 71 and light source 72.

2. The thermal therapy system according to claim 1, characterized in that it comprises an electric vacuum pump 70 which provides vacuum and creates a negative pressure in the range of −150 mmHg to −760 mmHg.

3. The thermal therapy system according to claim 1, characterized in that it comprises a light source 72 that radiates in the near-infrared wavelength for the photothermal ablation/coagulation method.

4. The thermal therapy system according to claim 3, characterized in that the light source 72 that radiates in the near-infrared wavelength for the photothermal ablation/coagulation method is an amplified spontaneous laser source, or a semiconductor laser source, or a fiber laser source, or a solid-state laser source.

5. The thermal therapy system according to claim 3, characterized in that the laser beam from the light source 72 that radiates in the near-infrared wavelength for the photothermal ablation/coagulation method has an optical waveguide to be easily transmitted to the 360-degree therapeutic apparatus 10 in which an optical waveguide array surrounds the recessed channel, or the 90-degree treatment apparatus using optical waveguides 30, or a 90-degree treatment apparatus using an array of optical waveguides coupled with polished ball-lensed tips 50, or a 360-degree treatment apparatus with a centered conical mirror 80, or a 0 to 360-degree active angle control therapeutic apparatus 110.

6. The thermal therapy system according to claim 5, characterized in that the optical waveguide transmitting the laser beam is a single-mode optical fiber, or a multi-mode optical fiber.

7. The thermal therapy according to claim 1, characterized in that the 360-degree therapeutic apparatus 10 in which an optical waveguide array surrounds the recessed channel, or the 90-degree therapeutic apparatus using optical waveguides 30, or a 90-degree therapeutic apparatus using an array of optical waveguides coupled with polished ball-lensed tips 50, or a 360-degree therapeutic apparatus with a centered conical mirror 80, or a 0 to 360-degree active angle control therapeutic apparatus 110 has an outer diameter in the range of 10 mm to 20 mm for appropriate intervention in the human gastrointestinal tract.

8. The thermal therapy system according to claim 1, characterized in that the 360-degree therapeutic apparatus 10 in which an optical waveguide array surrounds the recessed channel located therein comprises:
a top cover 11 that forms the top of the 360-degree therapeutic apparatus 10,
a lower cover 13 that forms the bottom of the 360-degree therapeutic apparatus 10,
a body with a recessed channel 12 that overlaps with the top cover 11 and lower cover 13, forms the whole of the 360-degree therapeutic apparatus 10, and has a circumferential row of holes for the negative pressure application,
an optical waveguide bundle that is located so as to have a circular array on the bottom of the recessed channel and allows the therapeutic laser beam to be transmitted to the 360-degree therapeutic apparatus 10.

9. The thermal therapy system according to claim 1, characterized in that the 90-degree therapeutic apparatus using optical waveguides 30 located therein comprises:
an upper body with a 90° opening 31 for the negative pressure applications, which forms the top of the 90-degree therapeutic apparatus using optical waveguides 30,
a lower cover 13 that forms the bottom of the 90-degree treatment apparatus using optical waveguides (30) and forms the whole of the 90-degree therapeutic apparatus using optical waveguides 30 by the overlap of the upper body with a 90° opening 31,
an optic waveguide bundle that is located on the bottom of the recessed channel in an arc shape of a 90° angle and allows the therapeutic laser beam to be transmitted to the 90-degree therapeutic apparatus using optical waveguides 30.

10. The thermal therapy system according to claim 8 or claim 9, characterized in that the optical waveguides located therein are a flat-ended optical fiber 14 or a tapered optical fiber 15.

11. An operation method of the thermal therapy system according to claim 8 or claim 9, characterized in that it comprises the following process steps:
pulling the target mucosal tissue with negative pressure into the body with a recessed channel 12 of the 360-degree therapeutic apparatus 10 in which an optical waveguide array surrounds the recessed channel, or into the opening on the upper body with a 90-degree opening 31 of the 90-degree therapeutic apparatus using optical waveguides 30,
transmitting the laser beam with optical waveguides located on the body to the 360-degree therapeutic apparatus 10 or 90-degree therapeutic apparatus using optical waveguides 30.

12. The thermal therapy system according to claim 1, characterized in that the 90-degree therapeutic apparatus using an array of optical waveguides coupled with polished ball-lensed tips 50 located therein comprises:
a d-shaped cylinder to fix the optical waveguides in place 51,
a glass ferrule 52 located on the d-shaped cylinder to fix the optical waveguides in place 51 as a protective,
a glass top cover with grooves 53 that fixes the d-shaped cylinder to fix the optical waveguides in place 51 and glass ferrule 52 and forms the top of the 90-degree therapeutic apparatus using an array of optical waveguides coupled with polished ball-lensed tips 50,
a lower body with a 90-degree opening 54 that forms the 90-degree therapeutic apparatus using an array of optical waveguides coupled with polished ball-lensed tips 50 by the overlap of the framework composed of a glass top cover with grooves 53, fixing the d-shaped cylinder to fix the optical waveguides in place 51 and glass ferrule 52 in place,
optical fibers coupled with polished ball-lensed tips 55 that are located in the d-shaped cylinder to fix the optical waveguides in place 51 so as to be two layers along the arc of 90° and transmits the therapeutic laser beam to the 90-degree therapeutic apparatus using an array of optical waveguides coupled with polished ball-lensed tips 50.

13. The thermal therapy system according to claim 12, characterized in that the optical waveguide coupled with ball lens in the 90-degree therapeutic apparatus design using an array of optical waveguides coupled with polished ball-lensed tips 50 is angled-polished.

14. An operation method of the thermal therapy system according to claim 12, characterized in that it comprises the following process steps:
pulling the target mucosal tissue with negative pressure to the opening on the lower body with a 90-degree opening 54,
transmitting the laser beam to the 90-degree therapeutic apparatus using an array of optical waveguides coupled with polished ball-lensed tips 50 by optical fibers coupled with polished ball-lensed tips 55 arranged in an arc to form a double-row in the d-shaped cylinder to fix the optical waveguides in place 51 within the glass ferrule 52 that is placed in the center of the glass top cover with grooves 53,
reflecting the laser beams directed by the ball lenses the glass ferrule 52 to the opening of the 90-degree therapeutic apparatus using an array of optical waveguides coupled with polished ball-lensed tips 50.

15. The thermal therapy system according to claim 1, characterized in that the 360-degree therapeutic apparatus with a centered conical mirror 80 located therein comprises:
a conical mirror 85 that is fixed in place in the center of the top cover and reflects the laser beam transmitted to the 360-degree therapeutic apparatus with a centered conical mirror 80,
a top cover for 360-degree reflection 81 that forms the top of the 360-degree therapeutic apparatus with a centered conical mirror 50,
a body with a recessed channel 12 that comprises a circumferential row of holes for negative pressure and forms the whole of the 360-degree therapeutic apparatus with a centered conical mirror 80 by the overlap of the top and bottom parts,
a lower-right cover for a single beam 83 that forms the bottom of the 360-degree therapeutic apparatus with a centered conical mirror 80 together with the lower-left cover for a single beam 82,
an optical waveguide coupled collimator (84) that is fixed in place in the center of the bottom part formed by the overlap of the lower-right cover for a single beam 83 and lower-left cover for a single beam 82 and collimates the therapeutic laser beam to be transmitted to the 360-degree therapeutic apparatus with a centered conical mirror 80.

16. An operation method of the thermal therapy system according to claim 15, characterized in that it comprises the following process steps:
pulling the target mucosal tissue with negative pressure to the body with a recessed channel 12,
using the optical waveguide coupled collimator 84 to transmit the laser beam to the 360-degree therapeutic apparatus with a centered conical mirror 80,
circumferentially reflecting the collimated laser beam by the conical mirror 85 fixed in place thereof on the top cover of the 360-degree therapeutic apparatus with a centered conical mirror 80,
positioning the circular plane formed by the reflected beam to the recessed channel of the 360-degree therapeutic apparatus with a centered conical mirror 80.

17. The thermal therapy system according to claim 1, characterized in that the 0 to 360-degree active angle control therapeutic apparatus 110 comprises:
an upper left cover for single-beam reflection 111 that forms the top of the 0 to 360-degree active angle control therapeutic apparatus 110 together with the upper right cover for single-beam reflection 112,
right-angle mirrors 115 that are fixed in place thereof on the top part formed by the overlap of the upper left cover for single-beam reflection 111 and the upper right cover for single-beam reflection 112 and reflect the laser beam, which is transmitted to the 0 to 360-degree active angle control therapeutic apparatus 110, vertically to the shaft rod end of the stepper motor,
a body with a recessed channel 12 that that comprises a circumferential row of holes for negative pressure and forms the whole of the 0 to 360-degree active angle control therapeutic apparatus 110 by the overlap of the top and bottom parts,
a lower left cover for single-beam transmission and scanning 113 that forms the bottom of the 0 to 360-degree active angle control therapeutic apparatus 110 together with the lower left cover for single-beam transmission and scanning 114,
an optical waveguide coupled collimator 84 that is fixed in place in the bottom part formed by the overlap of the lower left cover for single-beam transmission and scanning 113 and the lower left cover for single-beam transmission and scanning 114 and collimates the therapeutic laser beam to be transmitted to the 0 to 360-degree active angle control therapeutic apparatus 110,
a stepper motor 116 that is fixed in place in the bottom part formed by the overlap of the lower left cover for single-beam transmission and scanning 113 and the lower left cover for single-beam transmission and scanning 114 and driven by a driver board 130 for circumferential or local surface scanning of the laser beam.

18. The thermal therapy system according to claim 17, characterized in that it comprises a driver board 130 that increases the step resolution of the rotation motion of the stepper motor 116 to a maximum of 0.1 degrees for circumferential or local surface scanning of the laser beam.

19. The thermal therapy and imaging system according to claim 17, characterized in that it comprises a stepper motor 116 in which the rotational motion is actively controlled clockwise and counterclockwise by the driver board 130 for the stepper motor.

20. An operation method of the thermal therapy system according to claim 17, characterized in that it comprises the following process steps:
pulling the target mucosal tissue with negative pressure to the body with a recessed channel 12,
transmitting the laser beam to the 0 to 360-degree active angle control therapeutic apparatus 110 using the optical waveguide coupled collimator 84,
reflecting the collimated laser beam to another right-angle mirror 115 located to the tip of the shaft rod of the stepper motor 116 with the right-angle mirrors 115 fixed in place on the top cover of the 0 to 360-degree active angle control therapeutic apparatus 110,
performing the circumferential or local surface scanning of the laser beam by rotating the stepper motor 116.

21. The thermal therapy system according to claim 15 or claim 17, characterized in that it comprises an optical waveguide coupled collimator 84 that transmits the therapeutic laser beam to the 360-degree therapeutic apparatus with a centered conical mirror 80 or 0 to 360-degree active angle control therapeutic apparatus 110.

22. The thermal therapy system according to claim 21, characterized in that the optical waveguide coupled collimator 84 is a graded-index (GRIN)-lensed fiber collimator, or fiber-coupled aspherical lens collimator.

23. The thermal therapy system according to claim 15 or claim 17, characterized in that the conical mirror 85 that reflects the laser beam transmitted to the 360-degree therapeutic apparatus with a centered conical mirror 80, or right-angle mirrors 115 that reflects the laser beam, which is transmitted to the 0 to 360-degree active angle control therapeutic apparatus 110, vertically to the shaft rod end of the stepper motor has a protected gold or silver or aluminum coating.

24. The thermal therapy system according to claim 15 or claim 17, characterized in that the reflective surface of the conical mirror 85 that reflects the laser beam transmitted to the 360-degree therapeutic apparatus with a centered conical mirror 80, or right-angle mirror 115 that reflects the laser beam, which is transmitted to the 0 to 360-degree active angle control therapeutic apparatus 110, vertically to the shaft rod end of the stepper motor has a protective coating layer against scratching and oxidation.

25. The thermal therapy system according to claim 24, characterized in that the protective layer is silicon monoxide (SiO) or silicon dioxide (SiO₂).

26. The thermal therapy system according to claim 8, claim 12, claim 15 or claim 17, characterized in that the parts constituting the casing of the 360-degree therapeutic apparatus 10, or the 90-degree therapeutic apparatus using optical waveguides 30, or 90-degree therapeutic apparatus using an array of optical waveguides coupled with polished ball-lensed tips 50, or 360-degree therapeutic apparatus with a centered conical mirror 80, or 0 to 360-degree active angle control therapeutic apparatus 110 are made of a biocompatible material.

27. The thermal therapy system according to claim 8, claim 12, claim 15 or claim 17, characterized in that the biocompatible material used in the 360-degree therapeutic apparatus 10, or the 90-degree therapeutic apparatus using optical waveguides 30, or 90-degree therapeutic apparatus using an array of optical waveguides coupled with polished ball-lensed tips 50, or 360-degree therapeutic apparatus with a centered conical mirror 80, or body with a recessed channel 12 of the 0 to 360-degree active angle control therapeutic apparatus 110, or upper body with a 90-degree opening 31, or lower body with a 90-degree opening 54 is transparent and in particularly has an optical transparency in the range of 400 nm to 2000 nm.

28. The thermal therapy system according to claim 26 or claim 27, characterized in that the biocompatible material further has a light and workable form which has a high surface hardness, low water absorption and dimensional stability, and is resistant to scratches, chemicals, heat, ultraviolet rays, and atmospheric conditions.

29. A 0 to 360-degree active angle controlled thermal therapy and imaging system to be used in endoscopic medical diagnostic applications, endoscopic medical therapy applications and therapy photothermal (laser) ablation/coagulation therapy applications, characterized in that it comprises:
an electric vacuum pump 70 that creates a negative pressure necessary to pull the target tissue into the recessed channel of the therapeutic apparatus,
a manometer 71 that is connected in series to the electric vacuum pump 70 and continuously measures the negative pressure value,
a light source 72 that exposes the target tissue pulled into the recessed channel of the therapeutic apparatus to photothermal ablation/coagulation,
an optical (400 nm-2000 nm) imaging device 160 that performs the imaging of the target tissue pulled into the apparatus with negative pressure,
a 0 to 360-degree active angle control therapeutic and imaging apparatus 140, or MEMS mirror based 0 to 90-degree active angle control therapeutic and imaging apparatus 170, at least one of which is connected to an electric vacuum pump 70, manometer 71, light source 72 and an optical (400 nm-2000 nm) imaging device 160 to provide photothermal ablation therapy and imaging.

30. The thermal therapy and imaging system according to claim 29, characterized in that it comprises an electric vacuum pump 70 that provides vacuum and creates a negative pressure in the range of −150 mmHg to −760 mmHg.

31. The thermal therapy and imaging system according to claim 29, characterized in that it comprises a light source 72 that radiates in the near-infrared wavelength for the photothermal ablation/coagulation method or optical imaging.

32. The thermal therapy and imaging system according to claim 31, characterized in that the light source 72 that radiates in the near-infrared or visible wavelength for the photothermal ablation/coagulation method or optical imaging is an amplified spontaneous laser source, or a semiconductor laser source, or a fiber laser source, or a solid-state laser source.

33. The thermal therapy and imaging system according to claim 31, characterized in that it comprises an optical waveguide for easily transmitting the laser beam from the light source 72, which radiates in the near-infrared or visible wavelength, to the 0 to 360-degree active angle control therapeutic and imaging apparatus 140, or MEMS mirror based 0 to 90-degree active angle control therapeutic and imaging apparatus 170.

34. The thermal therapy and imaging system according to claim 33, characterized in that the optical waveguide transmitting the laser beam is a single-mode optical fiber, or a multi-mode optical fiber.

35. The thermal therapy and imaging system according to claim 29, characterized in that the diameter of the 0 to 360-degree active angle control therapeutic and imaging apparatus 140, or MEMS mirror based 0 to 90-degree active angle control therapeutic and imaging apparatus 170 is in the range of 10 mm to 20 mm for appropriate intervention in the human gastrointestinal tract.

36. The thermal therapy and imaging system according to claim 29, characterized in that the 0 to 360-degree active angle control therapeutic and imaging apparatus 140 comprises:
an upper left cover for double-beam reflection 141 that forms the top of the 0 to 360-degree active angle control therapeutic and imaging apparatus 140 together with the upper right cover for double-beam reflection 142,
right-angle mirrors 115 that is located on the top part formed by the overlap of the upper left cover for double-beam reflection 141 with the upper right cover for double-beam reflection 142 and transmits laser beams to the 0 to 360-degree active angle control therapeutic and imaging apparatus 140,
a knife-edge prism mirror 146 that is located on the top part formed by the overlap of the upper left cover for double-beam reflection 141 with the upper right cover for double-beam reflection 142 and transmits laser beams vertically to the tip of the shaft rod of the motor of the 0 to 360-degree active angle control therapeutic and imaging apparatus 140,
a body with a recessed channel 12 that forms the whole of the 0 to 360-degree active angle control therapeutic and imaging apparatus 140 by the overlap of the top and bottom parts,
a lower left cover for double-beam transmission and scanning 143 that forms the bottom of the 0 to 360-degree active angle control therapeutic and imaging apparatus 140 together with the lower right cover for double-beam transmission and scanning 144,
an optical waveguide coupled collimator 84 that is fixed in place in the bottom part formed by the overlap of the upper left cover for double-beam reflection 141 with the upper right cover for double-beam reflection 142 and collimates the therapeutic laser beam to be transmitted to the 0 to 360-degree active angle control therapeutic and imaging apparatus 140,
an optical waveguide coupled focusing lens 145 that is fixed in place in the bottom part formed by the overlap of the upper left cover for double-beam reflection 141 with the upper right cover for double-beam reflection 142 and focuses the imaging laser beam to be transmitted to the 0 to 360-degree active angle control therapeutic and imaging apparatus 140,
a stepper motor 116 that is fixed in place in the bottom part formed by the overlap of the upper left cover for double-beam reflection 141 with the upper right cover for double-beam reflection 142 and driven by a driver board 130 for circumferential or local surface scanning of the laser beam.

37. The thermal therapy and imaging system according to claim 36, characterized in that it comprises an optical waveguide coupled collimator 84 that transmits the therapeutic laser beam to the 0 to 360-degree active angle control therapeutic and imaging apparatus 140.

38. The thermal therapy and imaging system according to claim 37, characterized in that the optical waveguide coupled collimator 84 is a graded-index (GRIN)-lensed fiber collimator, or fiber-coupled aspherical lens collimator.

39. The thermal therapy and imaging system according to claim 36, characterized in that it comprises a driver board 130 that increases the step resolution of the rotational motion of the stepper motor 116 to a maximum of 0.1 degrees for circumferential or local surface scanning of the laser beam.

40. The thermal therapy and imaging system according to claim 36, characterized in that it comprises a stepper motor 116 in which the rotational motion is actively controlled clockwise and counterclockwise by the driver board 130 for the stepper motor.

41. An operation method of the thermal therapy and imaging system according to claim 36, characterized in that it comprises the following process steps:
pulling the target mucosal tissue with negative pressure to the body with a recessed channel 12, transmitting the laser beam to the 0 to 360-degree active angle control therapeutic and imaging apparatus 140 using the optical waveguide coupled collimator 84,
transmitting the imaging laser beam to the 0 to 360-degree active angle control therapeutic and imaging apparatus 140 using the optical waveguide coupled focusing lens 145,
directing the collimated and focused laser beams to the knife-edge prism mirror 146 that is fixed in place in the top cover of the 0 to 360-degree active angle control therapeutic and imaging apparatus 140 with the right-angle mirrors 115 that are fixed in place in the top cover,
reflecting the collimated and focused laser beams with to the knife-edge prism mirror 146 to another right-angle mirror 115 located to the tip of the shaft rod of the stepper motor 116,
performing the circumferential or local surface scanning of the laser beams by rotating the stepper motor 116.

42. The thermal therapy and imaging system according to claim 29, characterized in that the MEMS mirror based 0 to 90-degree active angle control therapeutic and imaging apparatus 170 comprises:
a back cover for single-beam reflection 171 that forms the back of the MEMS mirror based 0 to 90-degree active angle control therapeutic and imaging apparatus 170,
a front cover with a 90-degree opening 172 that forms the front of the MEMS mirror based 0 to 90-degree active angle control therapeutic and imaging apparatus 170 and the whole of the MEMS mirror based 0 to 90-degree active angle control therapeutic and imaging apparatus 170 by overlap with the back cover for single-beam reflection 171,
a MEMS mirror with a flexible printed circuit 173 that reflects the laser beam to the opening of the MEMS mirror based 0 to 90-degree active angle control therapeutic and imaging apparatus 170 for the surface scanning of the laser beam,
an optical waveguide coupled focusing lens 145 that transmits the therapeutic and imaging laser beams and the same to the MEMS mirror of the MEMS mirror based 0 to 90-degree active angle control therapeutic and imaging apparatus 170.

43. The thermal therapy and imaging system according to claim 42, characterized in that it comprises a flexible printed circuit board 190 which controls the MEMS mirror with a flexible printed circuit 173 for the laser surface scanning.

44. An operation method of the treatment and imaging system according to claim 42, characterized in that it comprises the following process steps:
pulling the target mucosal tissue with negative pressure into the opening on the front cover with a 90-degree opening 172,
transmitting the laser beam to the MEMS mirror based 0 to 90-degree active angle control therapeutic and imaging apparatus 170 using the optical waveguide coupled focusing lens 145,
reflecting the focused laser beam from the MEMS mirror with a flexible printed circuit 173 located above the optical focusing lens 145, in particularly to the opening of the MEMS mirror based 0 to 90-degree active angle control therapeutic and imaging apparatus 170,
performing 0 to 90-degree surface scanning by moving the micro-electronic mechanical system (MEMS) mirror controlled by a flexible printed circuit at certain angles.

45. The thermal therapy and imaging system according to claim 36 or claim 42, characterized in that it comprises an optical waveguide coupled focusing lens 145 that transmits the focused laser beam to the 0 to 360-degree active angle control therapeutic and imaging apparatus 140 or MEMS mirror based 0 to 90-degree active angle control therapeutic and imaging apparatus 170.

46. The thermal therapy and imaging system according to claim 45, characterized in that the optical waveguide coupled focusing lens 145 is a graded-index (GRIN)-lensed fiber collimator, or fiber-coupled aspherical lens collimator.

47. The thermal therapy and imaging system according to claim 36 or claim 42, characterized in that the right-angle mirror 115 that reflects laser beams transmitted to the 0 to 360-degree active angle control therapeutic and imaging apparatus 140, or the knife-edge prism mirror 146 that reflects laser beams, which is transmitted to the 0 to 360-degree active angle control therapeutic and imaging apparatus 140, vertically to the shaft rod end of the stepper motor, or the MEMS mirror with a flexible printed circuit 173 that reflects the laser beam, transmitted to the MEMS mirror based 0 to 90-degree active angle control therapeutic and imaging apparatus 170, to the opening of the apparatus has a protected gold or silver or aluminum coating.

48. The thermal therapy and imaging system according to claim 36 or claim 42, characterized in that the reflective surface of the right-angle mirror 115 that reflects laser beams transmitted to the 0 to 360-degree active angle control therapeutic and imaging apparatus 140, or the knife-edge prism mirror 146 that reflects laser beams, which is transmitted to the 0 to 360-degree active angle control treatment and imaging apparatus 140, vertically to the shaft rod end of the stepper motor, or the MEMS mirror with a flexible printed circuit 173 that reflects the laser beam, which is transmitted to the MEMS mirror based 0 to 90-degree active angle control therapeutic and imaging apparatus 170, to the opening of the apparatus has a protective coating layer against scratching and oxidation.

49. The thermal therapy and imaging system according to claim 48, characterized in that the protective coating is silicon monoxide (SiO) or silicon dioxide (SiO₂).

50. The thermal therapy system according to claim 36 or claim 42, characterized in that the parts constituting the casing of the 0 to 360-degree active angle control therapeutic and imaging apparatus 140 or the MEMS mirror based 0 to 90-degree active angle control therapeutic and imaging apparatus 170 are made of a biocompatible material.

51. The thermal therapy system according to claim 36 or claim 42, characterized in that the biocompatible material used in the 0 to 360-degree active angle control therapeutic and imaging apparatus 140 or the body with a recessed channel 12 of the MEMS mirror based 0 to 90-degree active angle control therapeutic and imaging apparatus 170, or the front cover with a 90-degree opening 172 is transparent and in particularly has an optical transparency in the range of 400 nm to 2000 nm.

52. The thermal therapy system according to claim 50 or claim 51, characterized in that the biocompatible material further a light and workable form which has a high surface hardness, low water absorption and dimensional stability, and is resistant to scratches, chemicals, heat, ultraviolet rays, and atmospheric conditions.

53. A 0 to 360-degree active angle controlled thermal therapy and imaging system to be used in endoscopic medical diagnostic applications, endoscopic medical therapy applications and radiofrequency ablation treatment applications, characterized in that it comprises:
an electric vacuum pump 70 that creates a negative pressure necessary to pull the target tissue into the recessed channel of the therapeutic and imaging apparatus,
a manometer 71 that is connected in series to the electric vacuum pump 70 and continuously measures the negative pressure value,
a radiofrequency generator 250 that exposes the target tissue pulled into the recessed channel of the apparatus to radiofrequency ablation,
an optical (400 nm-2000 nm) imaging device 160 that performs the imaging of the target tissue pulled into the apparatus with negative pressure,
a radiofrequency-based 360-degree therapeutic and optical imaging apparatus 200, or a radiofrequency-based 90-degree therapeutic and optical imaging apparatus 220, or a 90-degree therapeutic and optical imaging apparatus using the radiofrequency ablation method and a MEMS mirror 260, at least one of which is connected to an electric vacuum pump 70, manometer 71, radiofrequency generator 250, and an optical (400 nm-2000 nm) imaging device 160 to provide radiofrequency ablation therapy and imaging.

54. The thermal therapy and imaging system according to claim 53, characterized in that it comprises an electric vacuum pump 70 which provides vacuum and creates a negative pressure in the range of −150 mmHg to −760 mmHg.

55. The thermal therapy and imaging system according to claim 53, characterized in that the alternating current frequency generated by the radiofrequency generator 250 for the radiofrequency ablation method is in the range of 450 kHz to 500 kHz.

56. The thermal therapy and imaging system according to claim 53, characterized in that the diameter of the radiofrequency-based 360-degree therapeutic and optical imaging apparatus 200, or radiofrequency-based 90-degree therapeutic and optical imaging apparatus 220, or 90-degree therapeutic and optical imaging apparatus using the radiofrequency ablation method and a MEMS mirror 260 is in the range of 10 mm to 20 mm for appropriate intervention in the human gastrointestinal tract.

57. The thermal therapy and imaging system according to claim 53, characterized in that the radiofrequency-based 360-degree therapeutic and optical imaging apparatus 200 comprises:
a body with a 360-degree opening 201 that forms the whole of the radiofrequency-based 360-degree therapeutic and optical imaging apparatus 200,
a 360-degree electrode array 202 that transmits the alternating current for therapeutic purposes to the radiofrequency-based 360-degree therapeutic and optical imaging apparatus 200.

58. The thermal therapy and imaging system according to claim 53, characterized in that the radiofrequency-based 90-degree therapeutic and optical imaging apparatus 220 comprises:
a body with a 90-degree opening 221 that forms the whole of the radiofrequency-based 90-degree therapeutic and optical imaging apparatus 220,
a 90-degree electrode array 222 that transmits the alternating current for therapeutic purposes to the radiofrequency-based 90-degree therapeutic and optical imaging apparatus 220.

59. The thermal therapy and imaging system according to claim 57 or claim 58, characterized in that it comprises a radiofrequency-based 360-degree therapeutic and optical imaging apparatus 200 or a radiofrequency-based 90-degree therapeutic and optical imaging apparatus 220, which can be mounted at the distal end of the insertion tube of a conventional endoscopy device.

60. The thermal therapy and imaging system according to claim 57 or claim 58, characterized in that the 360-degree electrode array 202 or the 90-degree electrode array 222, which transmits the alternating current to the radiofrequency-based 360-degree therapeutic and optical imaging apparatus 200 or radiofrequency-based 90-degree therapeutic and optical imaging apparatus 220, is made of copper metal.

61. The thermal therapy and imaging system according to claim 57 or claim 58, characterized in that it comprises a 360-degree electrode array 202 or a 90-degree electrode array 222 in a flexible form, which may be located on the radiofrequency-based 360-degree therapeutic and optical imaging apparatus 200 or radiofrequency-based 90-degree therapeutic and optical imaging apparatus 220.

62. The thermal therapy and imaging system according to claim 57 or claim 58, characterized in that it comprises a 360-degree electrode array 202 which is located at the bottom of the opening in the body with a 360-degree opening 201 of the radiofrequency-based 360-degree therapeutic and optical imaging apparatus 200 or a 90-degree electrode array 222 which is located at the bottom of the opening in the body with a 90-degree opening 221 of the radiofrequency-based 90-degree therapeutic and optical imaging apparatus 220.

63. An operation method of the thermal therapy and imaging system according to claim 57 or claim 58, characterized in that it comprises the following process steps:
pulling the target mucosal tissue into the opening in the body with a 360-degree opening 201 or the body with a 90-degree opening 221 by a negative pressure,
transmitting the alternating current at a level of radiofrequency energy to the radiofrequency-based 360-degree therapeutic and optical imaging apparatus 200 using electrodes or the radiofrequency-based 90-degree therapeutic and optical imaging apparatus 220 using electrodes.

64. The thermal therapy and imaging system according to claim 53, characterized in that the 90-degree therapeutic and optical imaging apparatus using the radiofrequency ablation method and a MEMS mirror 260 comprises:
a back cover for single-beam reflection 171 that forms the back of the 90-degree therapeutic and optical imaging apparatus using the radiofrequency ablation method and a MEMS mirror 260,
a front cover with a 90-degree opening 172 that forms the whole of the 90-degree therapeutic and optical imaging apparatus using the radiofrequency ablation method and a MEMS mirror 260 by the overlap with the back cover for single-beam reflection 171,
a MEMS mirror with a flexible printed circuit 173 that reflects the laser beam to the opening of the 90-degree therapeutic and optical imaging apparatus using the radiofrequency ablation method and a MEMS mirror 260 for surface scanning of the laser beam,
an optical waveguide coupled focusing lens 145 that transmits the focused therapeutic or imaging laser beam to the 90-degree therapeutic and optical imaging apparatus using the radiofrequency ablation method and a MEMS mirror 260,
a 90-degree electrode array 222 that transmits the therapeutic alternating current to the 90-degree therapeutic and optical imaging apparatus using the radiofrequency ablation method and a MEMS mirror 260 for radiofrequency ablation.

65. The thermal therapy and imaging system according to claim 64, characterized in that it comprises a flexible printed circuit board 190 that controls the MEMS mirror with a flexible printed circuit 173 used for the laser surface scanning.

66. The thermal therapy and imaging system according to claim 64, characterized in that it comprises a light source 72 that radiates in the near-infrared or visible wavelength for the photothermal ablation/coagulation method or optical imaging.

67. The thermal therapy and imaging system according to claim 66, characterized in that the light source 72 that radiates in the near-infrared or visible wavelength for the photothermal ablation/coagulation method or optical imaging is an amplified spontaneous laser source, or a semiconductor laser source, or a fiber laser source, or a solid-state laser source.

68. The thermal therapy and imaging system according to claim 66, characterized in that it comprises an optical waveguide for easily transmitting the laser beam from the light source 72, which radiates in the near-infrared or visible wavelength, to the 90-degree therapeutic and optical imaging apparatus using the radiofrequency ablation method and a MEMS mirror 260.

69. The thermal therapy and imaging system according to claim 68, characterized in that the optical waveguide transmitting the laser beam is a single-mode optical fiber, or a multi-mode optical fiber.

70. The thermal therapy and imaging system according to claim 64, characterized in that it comprises an optical waveguide coupled focusing lens 145 for transmitting the therapeutic or focusing the imaging laser beam to the 90-degree therapeutic and optical imaging apparatus using the radiofrequency ablation method and a MEMS mirror 260.

71. The thermal therapy and imaging system according to claim 70, characterized in that the optical waveguide coupled focusing lens 145 is a graded-index (GRIN)-lensed fiber collimator, or fiber-coupled aspherical lens collimator.

72. The thermal therapy and imaging system according to claim 64, characterized in that the MEMS mirror with a flexible printed circuit 173, which reflects the laser beam transmitted to the 90-degree therapeutic and optical imaging apparatus using the radiofrequency ablation method and a MEMS mirror 260 to the opening of the apparatus, has a protected gold or silver or aluminum coating.

73. The thermal therapy and imaging system according to claim 64, characterized in that the reflective surface of the MEMS mirror with a flexible printed circuit 173, which reflects the laser beam transmitted to the 90-degree therapeutic and optical imaging apparatus using the radiofrequency ablation method and a MEMS mirror 260 to the opening of the apparatus, has a protective coating layer against scratching and oxidation.

74. The thermal therapy and imaging system according to claim 73, characterized in that the protective layer is silicon monoxide (SiO) or silicon dioxide (SiO2).

75. The thermal therapy and imaging system according to claim 64, characterized in that the 90-degree electrode array 222 which transmits the alternating current to the 90-degree therapeutic and optical imaging apparatus using the radiofrequency ablation method and a MEMS mirror 260 is made of copper metal.

76. The thermal therapy and imaging system according to claim 64, characterized in that it comprises a 90-degree electrode array 222 in a flexible form which is located on the 90-degree therapeutic and optical imaging apparatus using the radiofrequency ablation method and a MEMS mirror 260.

77. The thermal therapy and imaging system according to claim 64, characterized in that it comprises a 90-degree electrode array 222 which is located at the bottom of the opening in the body with a 90-degree opening 221 of the 90-degree therapeutic and optical imaging apparatus using the radiofrequency ablation method and a MEMS mirror 260.

78. An operation method of the thermal therapy and diagnostic imaging system according to claim 64, characterized in that it comprises the following process steps:
pulling the target mucosal tissue into the opening of the front cover with a 90-degree opening 172 by a negative pressure,
transmitting the laser beam using an optical waveguide coupled focusing lens 145 to the 90-degree therapeutic and optical imaging apparatus using the radiofrequency ablation method and a MEMS mirror 260,
reflecting the focused and transmitted laser beam from the MEMS mirror with a flexible printed circuit 173 located above the optical waveguide coupled focusing lens 145, in particularly to the opening of the 90-degree therapeutic and optical imaging apparatus using the radiofrequency ablation method and a MEMS mirror 260,
performing 0° to 90° surface scanning by moving the micro-electronic mechanical system (MEMS) mirror controlled by a flexible printed circuit at certain angles,
use of electrodes to transmit the alternating current at a level of radiofrequency energy to the opening of the 90-degree therapeutic and optical imaging apparatus using the radiofrequency ablation method and a MEMS mirror 260.

79. The thermal therapy and imaging system according to claim 57, claim 58 or claim 64, characterized in that the parts constituting the casing of the radiofrequency-based 360-degree therapeutic and optical imaging apparatus 200, or the radiofrequency-based 90-degree therapeutic and optical imaging apparatus 220, or the 90-degree therapeutic and optical imaging apparatus using the radiofrequency ablation method and a MEMS mirror 260 are made of a biocompatible material.

80. The thermal therapy and imaging system according to claim 57, claim 58 or claim 64, characterized in that the biocompatible material used in the radiofrequency-based 360-degree therapeutic and optical imaging apparatus 200, or the radiofrequency-based 90-degree therapeutic and optical imaging apparatus 220, or the 90-degree therapeutic and optical imaging apparatus using the radiofrequency ablation method and a MEMS mirror 260, or the body with a 360-degree opening 201, or the body with a 90-degree opening 221, or the front cover with a 90-degree opening 172 is transparent and in particularly, has an optical transmittance in the range of 400 nm to 2000 nm.

81. The thermal therapy system according to claim 79 or claim 80, characterized in that the biocompatible material further has a light and workable form which has a high surface hardness, low water absorption and dimensional stability, and is resistant to scratches, chemicals, heat, ultraviolet rays, and atmospheric conditions.
